(19) United States
(12) Patent Application Publication
(10) Pub. No.: US 2007/0021450 A1
(43) Pub. Date: Jan. 25, 2007

(54) Indanylamino uracils and their use as antioxidants and neuroprotectants

(76) Inventors: Benjamin Sklarz, Petach-Tikva (IL); Eliezer Falb, Givataim (IL); Gyorgy Toth, Nyiregyhaza (HU); Yaacov Herzig, Raanana (IL); Jeffrey Sterling, Jerusalem (IL)

Correspondence Address: COOPER & DUNHAM, LLP, 1185 AVENUE OF THE AMERICAS, NEW YORK, NY, 10036, US

(21) Appl. No.: 11/182,452

(22) Filed: Jul. 15, 2005

Related U.S. Application Data
(60) Provisional application No. 60/588,477, filed on Jul. 16, 2004.

(51) Int. Cl. A61K 31/513; C07D 239/545

(52) U.S. Cl. 514/269; 544/309

(57) ABSTRACT

Disclosed are compounds having the structure: 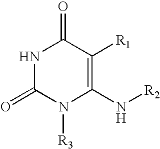 wherein,
R₁ is H, NH₂, NH—(C₁-C₄)alkyl, or N—[(C₁-C₄)alkyl]₂;
R₂ and R₃ are each independently H, (C₁-C₄)alkyl, or 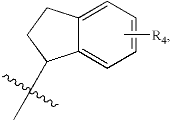
wherein R₄ is H, (C₁-C₄)alkyl, halogen, hydroxy, (C₁-C₁₀)alkoxy, cyano, nitro, —NR₅R₆, or —OCONR₇R₈;
   wherein R₅ and R₆ are each independently H, or a substituted or unsubstituted (C₁-C₄)alkyl; and
   wherein R₇ and R₈ are each independently H, or substituted or unsubstituted (C₁-C₄)alkyl, or (C₁-C₁₀)aryl; and
wherein only one of R₂ and R₃ is H,
   enantiomers, tautomers, and pharmaceutically acceptable salts of the compounds, pharmaceutical compositions containing such compounds or salts, and processes for their preparation. The subject invention also provides methods of alleviating symptoms of neurologic and inflammatory disorders, methods of preventing oxidation of lipids, proteins, or deoxyribonucleic acids on a cellular level, and methods of protecting human red blood cells from lysis by O₂ radicals.

This application claims benefit of U.S. Provisional Application No. 60/588,477, filed Jul. 16, 2004, the contents of which are hereby incorporated by reference.

Throughout this application various publications are referenced in parenthesis. The disclosures of these publications in their entireties are hereby incorporated by reference into this application in order to more fully describe the state of the art to which this invention pertains.

### BACKGROUND OF THE INVENTION

The role of nitric oxide, and particularly its interaction with the reactive oxygen species (ROS) pathways via the superoxide anion, is at the center of current interest (C. Szabo, Brain Res. Bull. (1996) 41:131). The interaction of NO with a superoxide radical (O₂⁻) leads to the formation of peroxynitrite (ONOO⁻). This reaction is extremely fast. Therefore, the concentration of peroxynitrite in a cell depends upon the concentrations of superoxide and NO in the cell (M. F. Beal, Curr. Opin. Neurobiol. (1996) 6:661). At physiologic pH, peroxynitrite has a half-life of 0.9 s, allowing it to diffuse over several cell diameters and cause cell damage by oxidizing lipids, proteins and DNA before it degrades.

Recent findings have shown that peroxynitrite reacts with carbon dioxide (CO₂) to form an unstable nitrosuperoxycarbonate adduct (O+NOOCO₂⁻) that appears to rearrange to give a nitrocarbonate anion (O₂NOCO₂⁻), which may serve as the proximal oxidant in biological systems (R. M. Uppu et al., Arch. Biochem. Biophys. (1996) 327:335). This unstable intermediate can produce one- and two-electron oxidations as well as electrophilic nitration. Nitration reactions result in the production of nitrotyrosine, which is used as a specific biochemical marker for peroxynitrite-mediated damage in vivo.

The nitric oxide discussed above is produced specifically by neuronal nitric oxide synthase (nNOS). Inhibition of nNOS in mice produced inhibition of MPTP neurotoxicity in mice and an associated decrease in striatal nitrotyrosine (J. B. Schultz et al., J. Neurochem. (1995) 64:936).

Many inflammatory, degenerative and atrophic disorders in humans are thought to be autoimmune disorders (Merck Manual, p. 1062). In acute CNS inflammatory conditions, and in autoimmune and chronic neurologic disorders (e.g. MS, AD), nitric oxide is also associated with inducible nitric oxide synthetase (iNOS) and seems to act via peroxynitrite (C. Szabo, Brain Res. Bull. (1996) 41:131).

Oxidative stress has been proposed as a pathogenic mechanism in Alzheimer's disease (AD) because nitrotyrosine, the product of attack by peroxynitrite, was detected in the neurofibrillary tangles (P. F. Good et al., Am. J. Pathol. (1996) 149:21). Oxidative stress may also contribute to neuronal degeneration and death in disorders ranging from ischemic stroke to Alzheimer's and Parkinson's to age related macular degeneration to amyotrophic lateral sclerosis (M. P. Mattson et al., J. Neurosci. Res. (1997) 49: 681), disorders in which NO, via peroxynitrite, plays a key role.

Oxidative stress has also been implicated in inflammatory bowel disease (Lih-Brody, L. et al. Digestive Diseases and Sciences. (1996) 41(10):2078) as well as in rheumatoid arthritis (M. Y. Cimen et al., Clinical-Rheumatology. (2000) 19(4):275) and in multiple sclerosis (Calabrese, V. et al. International Journal of Clinical Pharmacology Research (1994) 14(4):119).

Several strategies for conferring neuroprotection have been developed which target the complex neurochemical processes which follow neuronal malfunction. Older approaches (reviewed by N. G. Wahlgren, in R. Green, “International Review of Neurobiology: Neuroprotective Agents and Cerebral Ischemia”, Vol. 40, Academic Press, 1997) including closure of calcium channels (with calcium antagonists), inhibition of glutamate release, and antagonism to NMDA and GABA agonism have not led to any remarkable treatments. With the recent emphasis on the role of reactive oxygen species (ROS) and of the nitrogen oxyanion species, the focus of possible treatments has now shifted to antioxidant and free radical scavengers (K. Hensley et al., in “Neuroinflammation: mechanisms and management” (Ed: P. L. Wood), Humana Press Inc., 1997).

Uric acid is one of several low molecular weight antioxidants in the brain (reviewed by E. Shohami et al., J. Cereb. Blood Flow and Metab. (1997) 17:1007). It was recently shown that peroxynitrite is associated with lesions observed in multiple sclerosis (D. C. Hooper et al., Proc. Natl. Acad. Sci., USA, (1997) 94:2528) and that uric acid prevents virtually all the clinical symptoms of EAE, an accepted animal model of autoimmune disorders such as MS, while a statistical survey demonstrated the mutual exclusivity of MS and gout (hyperuricaemia) (D. C. Hooper et al., Proc. Natl. Acad. Sci., USA, (1998) 95:675). Uric acid was also shown to protect neurons against apoptosis induced by FeSO₄ and Aβ(25-35), and suppresses peroxynitrite accumulation (M. P. Mattson et al., J. Neurosci. Res. (1997) 49:681). Uric acid also protects ascorbic acid in the blood from oxidation, by sequestering Fe ions as a complex (K. J. A. Davies et al., Biochem. J. (1986) 235:747).

The present invention relates to derivatives of uracil, 5-aminouracil and 5,6-diaminouracils, comprising an indanyl functionality, which can be substituted, attached either to the N1 ring nitrogen of the uracil nucleus or to the nitrogen of the 6-amino group. The indanyl moiety is incorporated into the compounds of the invention primarily in order to increase lipophilicity and blood-brain-barrier (BBB) penetrability. Substituents on the aromatic ring which contain a basic amino moiety may be readily converted to their acid addition salts, thereby resulting in compounds with improved solubility in water.

### SUMMARY OF THE INVENTION

The subject invention provides a compound having the structure: 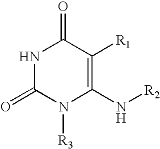
wherein,
R₁ is H, NH₂, NH—(C₁-C₄)alkyl, or N—[(C₁-C₄)alkyl]₂;
R₂ and R₃ are each independently H, (C₁-C₄)alkyl, or 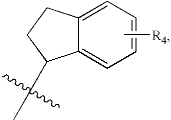
   wherein R₄ is H, (C₁-C₄)alkyl, halogen, hydroxy, (C₁-C₁₀)alkoxy, cyano, nitro, —NR₅R₆, or —OCONR₇R₈;
      wherein R₅ and R₆ are each independently H, or a substituted or unsubstituted (C₁-C₄)alkyl; and
      wherein R₇ and R₈ are each independently H, or substituted or unsubstituted (C₁-C₄)alkyl, or (C₁-C₁₀)aryl; and
   wherein only one of R₂ and R₃ is H,
      or an enantiomer, or tautomer, or a pharmaceutically acceptable salt of the compound.

The subject invention also provides a compound having the structure: 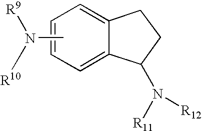
wherein,
   R₉ and R₁₀ are each independently a substituted or unsubstituted (C₁-C₄)alkyl; and
   R₁₁ and R₁₂ are each independently H or a substituted or unsubstituted (C₁-C₄)alkyl,
      or an enantiomer, or a tautomer, or a pharmaceutically acceptable salt thereof.

The subject invention further provides a method of treating a subject suffering from a neurologic disorder or an autoimmune disorder, comprising administering to the subject a therapeutically effective amount of any one of the disclosed compounds so as to thereby treat the subject.

The subject invention also provides a method of treating a subject afflicted with an inflammatory disorder caused by the presence of reactive oxidative species, comprising administering to the subject a therapeutically effective amount of any one of the disclosed compounds so as to thereby treat the subject.

The subject invention further provides a method of preventing the oxidation of lipids, proteins or deoxyribonucleic acid in a cell, comprising contacting the cells with any one of the disclosed compounds.

The subject invention further provides a method of preventing lysis of human red blood cells by O₂ radicals, comprising contacting the cells with any one of the disclosed compounds.

The subject invention also provides a pharmaceutical composition comprising any one of the disclosed compounds and a pharmaceutically acceptable carrier.

The subject invention further provides a process for the manufacture of a pharmaceutical composition comprising admixing any one of the disclosed compounds with a pharmaceutically acceptable carrier.

The subject invention also provides a packaged pharmaceutical composition for treating Alzheimer's disease, Parkinson's disease, multiple sclerosis, amyotrophic lateral sclerosis, age related macular degeneration or inflammation which is caused by the presence of peroxynitrite in a subject comprising a pharmaceutical composition of any one of the disclosed compounds, and instructions for using the disclosed composition for treating Alzheimer's disease, Parkinson's disease, multiple sclerosis, amyotrophic lateral sclerosis, age related macular degeneration or the inflammation which is caused by the presence of peroxynitrite in the subject.

The subject invention further provides a process for manufacturing a compound having the structure: 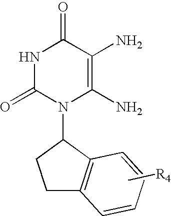
wherein R₄ is H, (C₁-C₄)alkyl, halogen, hydroxy, (C₁-C₁₀)alkoxy, cyano, nitro, —NR₅R₆, or —OCONR₇R₈;
   wherein R₅ and R₆ are each independently H, or a substituted or unsubstituted (C₁-C₄)alkyl; and
   wherein R₇ and R₈ are each independently H, or substituted or unsubstituted (C₁-C₄)alkyl, or (C₁-C₁₀) aryl,
      comprising reacting 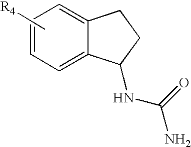
      with a cyclization agent to produce the compound.

The subject invention also provides a process of manufacturing a compound having the structure: 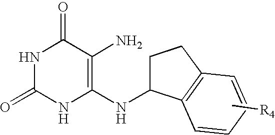
wherein R₄ is H, (C₁-C₄)alkyl, halogen, hydroxy, (C₁-C₁₀)alkoxy, cyano, nitro, —NR₅R₆, or —OCONR₇R₈;
   wherein R₅ and R₆ are each independently H, or a substituted or unsubstituted (C₁-C₄)alkyl; and
   wherein R₇ and R₈ are each independently H, or substituted or unsubstituted (C₁-C₄)alkyl, or (C₁-C₁₀) aryl,
      comprising reacting 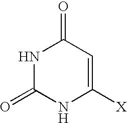
      wherein X is Cl or NH₂, with 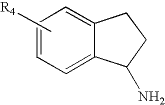
         in a high boiling solvent to produce the compound.

The subject invention further provides a process of manufacturing a compound having the structure: 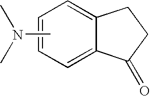
comprising reacting a compound having the structure 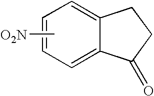
with paraformaldehyde and hydrogen over a palladium catalyst to produce the compound.

### DETAILED DESCRIPTION OF THE FIGURES

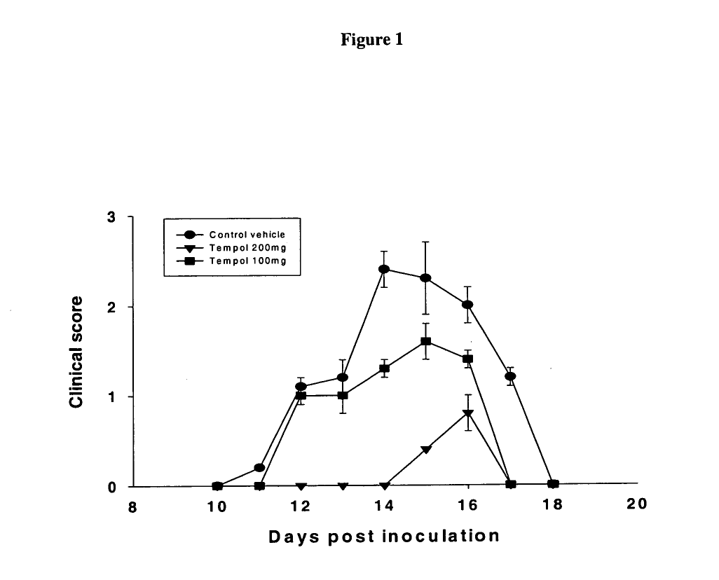

FIG. 1 shows the results of treatment of EAE in mice with 4-hydroxy-2,2,6,6-tetramethyl-piperidine-1-oxyl (TEMPOL).
-•- indicates the control group;
-▾- indicates group treated with Tempol 200 mg
-▪- indicates group treated with Tempol 100 mg.

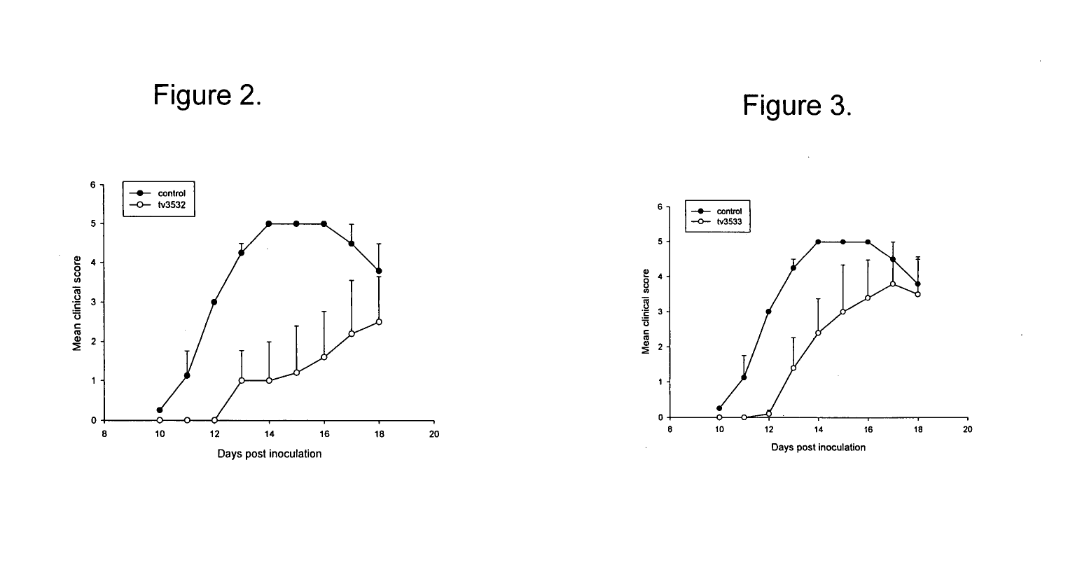

FIG. 2 shows the effect of compound 6 on EAE in PLP-challenged mice.
-•- indicates the control group
-∘- indicates the group treated with compound 6 (50 mg/kg/day)

FIG. 3 shows the effect of compound 7 on EAE in PLP-challenged mice.
-•- indicates the control group
-∘- indicates the group treated with compound 7 (50 mg/kg/day)

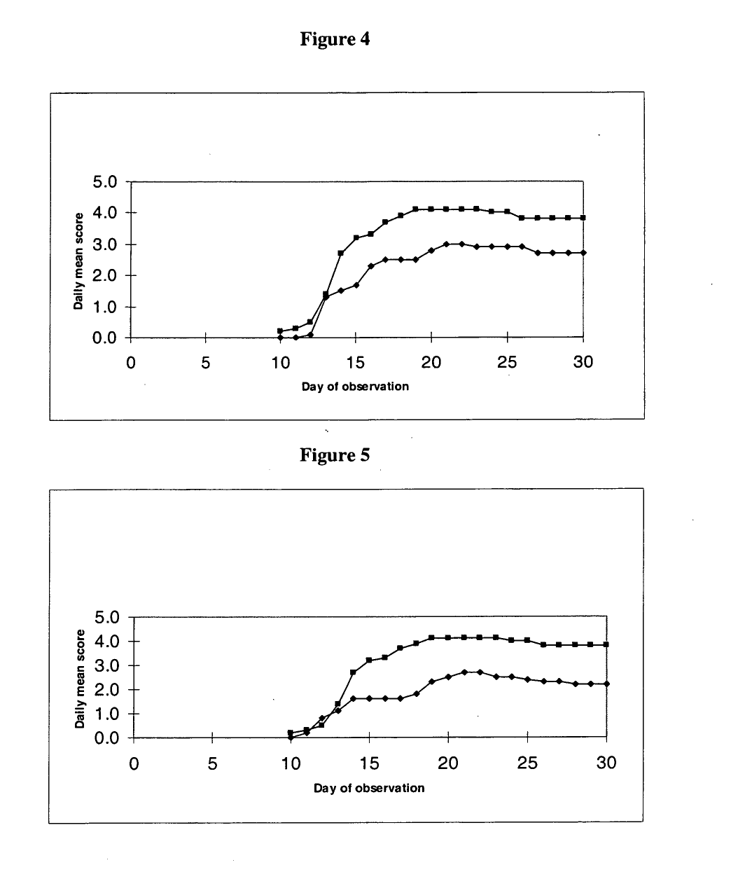

FIG. 4 shows the effect of compound 6 on EAE in CSJL mice.
-▪- indicates control group
-♦- indicates group treated with Compound 6 (50 mg/kg)

FIG. 5 shows the effect of compound 6 on EAE in CSJL mice.
-▪- indicates control group
-♦- indicates group treated with compound 6 (25 mg/kg×2)

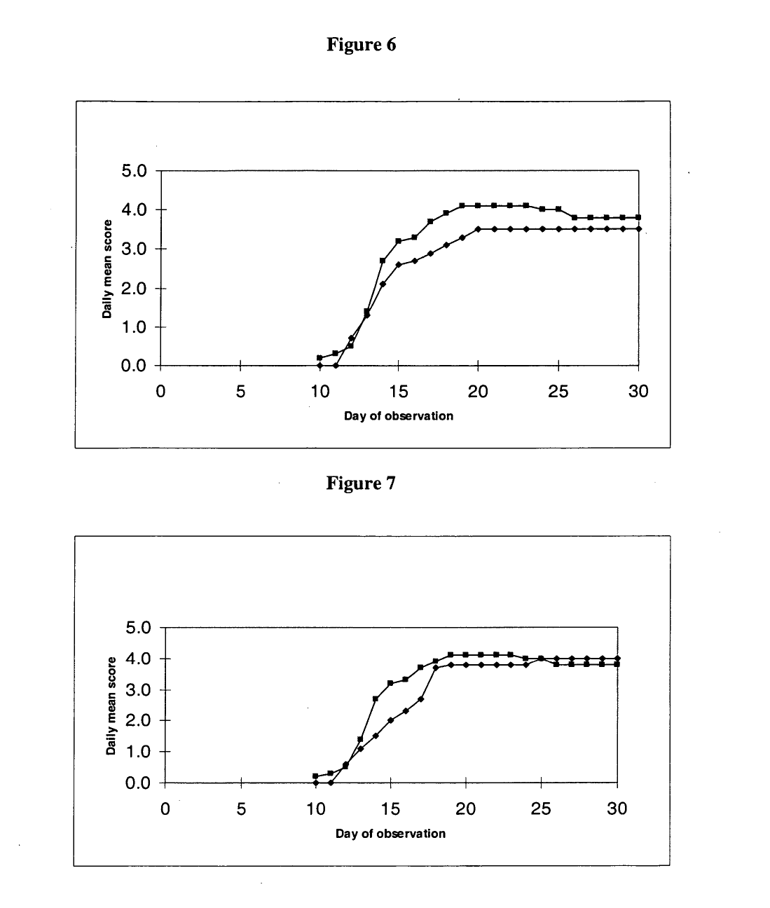

FIG. 6 shows the effect of compound 7 on EAE in CSJL mice.
-▪- indicates control group
-♦- indicates group treated with Compound 7 (50 mg/kg)

FIG. 7 shows the effect of compound 7 on EAE in CSJL mice.
-▪- indicates control group
-♦- indicates group treated with compound 7 (25 mg/kg×2)

### DETAILED DESCRIPTION OF THE INVENTION

The subject invention provides a compound having the structure: 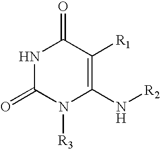
wherein,
R₁ is H, NH₂, NH—(C₁-C₄)alkyl, or N—[ (C₁-C₄)alkyl]₂;
R₂ and R₃ are each independently H, (C₁-C₄)alkyl, or 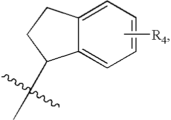
   wherein R₄ is H, (C₁-C₄)alkyl, halogen, hydroxy, (C₁-C₁₀)alkoxy, cyano, nitro, —NR₅R₆, or —OCONR₇R₈;
      wherein R₅ and R₆ are each independently H, or a substituted or unsubstituted (C₁-C₄)alkyl; and
      wherein R₇ and R₈ are each independently H, or substituted or unsubstituted (C₁-C₄)alkyl, or (C₁-C₁₀)aryl; and
wherein only one of R₂ and R₃ is H, or an enantiomer, or a tautomer, or a pharmaceutically acceptable salt thereof.

In an embodiment of the compound, R₁ is H or NH₂; R₂ and R₃ are each independently H, or 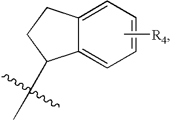
wherein R₄ is H, (C₁-C₄) alkyl, halogen, hydroxy, (C₁-C₁₀)alkoxy, cyano, nitro, —NR₅R₆, or —OCONR₇R₈;
wherein R₅ and R₆ are each independently H, or a substituted or unsubstituted (C₁-C₄)alkyl; and
wherein R₇ and R₈ are each independently H, or substituted or unsubstituted (C₁-C₄)alkyl, or (C₁-C₁₀)aryl; and
wherein only one of R₂ and R₃ is H.

In an additional embodiment, the compound has the structure: 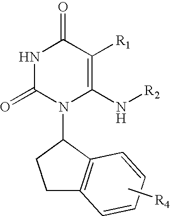
wherein
   R₁ is H or NH₂;
   R₂ is H; and
   R₄ is H, (C₁-C₄)alkyl, halogen, hydroxy, (C₁-C₁₀)alkoxy, cyano, nitro, —NR₅R₆, or —OCONR₇R₈;
      wherein R₅ and R₆ are each independently H, or a substituted or unsubstituted (C₁-C₄) alkyl; and
      wherein R₇ and R₈ are each independently H, or substituted or unsubstituted (C₁-C₄)alkyl, or (C₁-C₁₀)aryl.

In another embodiment, the compound has the structure:

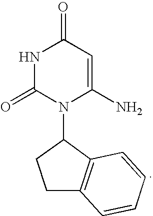

In another embodiment, the compound has the structure:

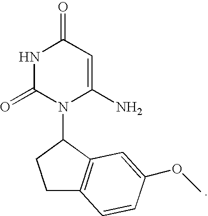

In yet another added embodiment, the compound has the structure:

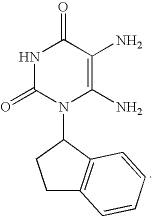

In a further embodiment, the compound has the structure: 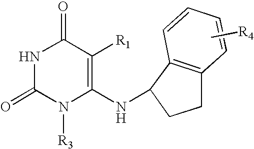
wherein
   R₁ is H or NH₂;
   R₃ is H; and
   R₄ is H, (C₁-C₄) alkyl, halogen, hydroxy, (C₁-C₁₀)alkoxy, cyano, nitro, —NR₅R₆, or —OCONR₇R₈;
      wherein R₅ and R₆ are each independently H, or a substituted or unsubstituted (C₁-C₄)alkyl; and
      wherein R₇ and R₈ are each independently H, or substituted or unsubstituted (C₁-C₄)alkyl, or (C₁-C₁₀)aryl.

In one embodiment, the compound has the structure:

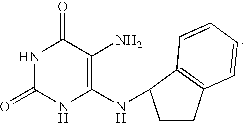

In another embodiment, the compound has the structure:

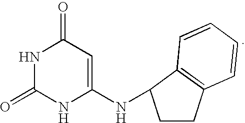

In a further embodiment, the compound has the structure:

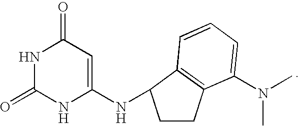

In yet another embodiment, the compound is the hydrochloride salt of:

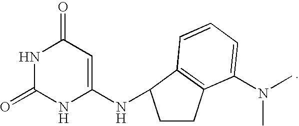

In another embodiment, the compound has the structure:

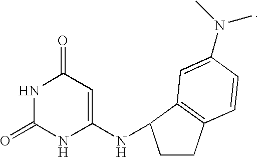

In a further embodiment, the compound is the hydrochloride salt of:

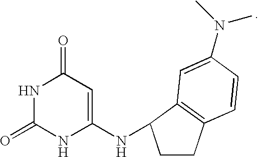

In another embodiment, the compound has the structure:

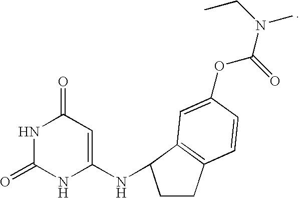

In yet another embodiment, the compound has the structure:

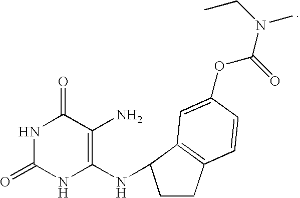

The subject invention also provides a compound having the structure: 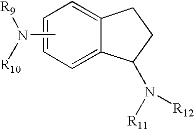
wherein,
   R₉ and R₁₀ are each independently a substituted or unsubstituted (C₁-C₄)alkyl; and
   R₁₁ and R₁₂ are each independently H or a substituted or unsubstituted (C₁-C₄)alkyl,
      or an enantiomer, or a tautomer, or a pharmaceutically acceptable salt thereof. In one embodiment, R₉ and R₁₀ are both methyl and R₁₁ and R₁₂ are both H.

The subject invention also provides a method of treating a subject suffering from a neurologic disorder or an autoimmune disorder, comprising administering to the subject a therapeutically effective amount of any one of the disclosed compounds so as to thereby treat the subject. In one embodiment, the subject suffers from a neurologic disorder. The neurologic disorder may be any one of Alzheimer's disease, Parkinson's disease, amyotrophic lateral sclerosis or age related macular degeneration. In another embodiment, the subject suffers from an autoimmune disorder. The autoimmune disorder may be multiple sclerosis.

The subject invention further provides a method of treating a subject afflicted with an inflammatory disorder caused by the presence of reactive oxidative species, comprising administering to the subject a therapeutically effective amount of any one of the disclosed compounds so as to thereby treat the subject. In one embodiment, the inflammatory disorder is caused by the presence of peroxynitrite in the subject. The inflammatory disorder may be an inflammatory bowel disease, or rheumatoid arthritis.

The subject invention also provides a method of preventing the oxidation of lipids, proteins or deoxyribonucleic acid in a cell, comprising contacting the cell with any one of the disclosed compounds.

The subject invention further provides a method of preventing lysis of human red blood cells by O₂ radicals, comprising contacting the cells with any one of the disclosed compounds.

The subject invention also provides a pharmaceutical composition comprising any one of the disclosed compounds and a pharmaceutically acceptable carrier.

Furthermore, the subject invention provides a process for the manufacture of a pharmaceutical composition comprising admixing any one of the disclosed compounds with a pharmaceutically acceptable carrier.

The subject invention provides a packaged pharmaceutical composition for treating Alzheimer's disease, Parkinson's disease, multiple sclerosis, amyotrophic lateral sclerosis, age related macular degeneration or inflammation which is caused by the presence of peroxynitrite in a subject comprising:
(a) the pharmaceutical composition of any one of the disclosed compounds; and
(b) instructions for using the composition for treating Alzheimer's disease, Parkinson's disease, multiple sclerosis, amyotrophic lateral sclerosis, age related macular degeneration or the inflammation which is caused by the presence of peroxynitrite in the subject.

The subject invention further provides a process for manufacturing a compound having the structure: 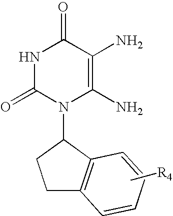
wherein R₄ is H, (C₁-C₄)alkyl, halogen, hydroxy, (C₁-C₁₀)alkoxy, cyano, nitro, —NR₅R₆, or —OCONR₇R₈;
wherein R₅ and R₆ are each independently H, or a substituted or unsubstituted (C₁-C₄)alkyl; and
wherein R₇ and R₈ are each independently H, or substituted or unsubstituted (C₁-C₄)alkyl, or (C₁-C₁₀)aryl,
   comprising reacting 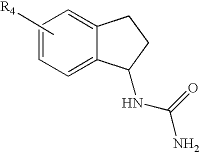
   with a cyclization agent to produce the compound.

An embodiment, of the above process further comprises the steps of:
(a) reacting 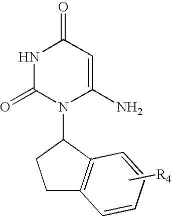
with isoamyl nitrite and HCl to produce 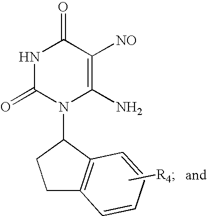
(b) reacting the product of step (a) with a reducing agent to produce

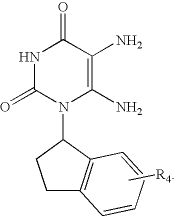

In another embodiment of the above process, the cyclization agent is ethyl cyanoacetate. In yet another embodiment of the above process, the reducing agent in step (b) is sodium dithionite.

The subject invention also provides a process of manufacturing a compound having the structure: 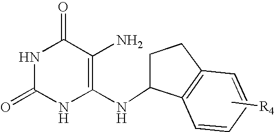
wherein R₄ is H, (C₁-C₄)alkyl, halogen, hydroxy, (C₁-C₁₀)alkoxy, cyano, nitro, —NR₅R₆, or —OCONR₇R₈;
   wherein R₅ and R₆ are each independently H, or a substituted or unsubstituted (C₁-C₄)alkyl; and
   wherein R₇ and R₈ are each independently H, or substituted or unsubstituted (C₁-C₄)alkyl, or (C₁-C₁₀)aryl,
comprising reacting 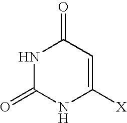
wherein X is Cl or NH₂, with 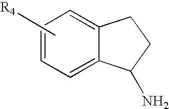
in a high boiling solvent to produce the compound.

An embodiment of the immediately preceding process further comprises the steps of:
(a) reacting 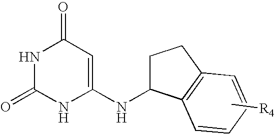
with isoamyl nitrite and HCl to produce 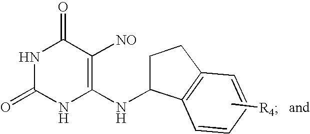
(b) reacting the product of step (a) with a reducing agent to produce:

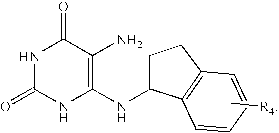

In another embodiment of this process immediately described above, the high boiling solvent is ethylene glycol dimethyl ether or DMSO. In yet another embodiment of this process, the reducing agent in step (b) is sodium dithionite.

The subject invention further provides a process of manufacturing a compound having the structure: 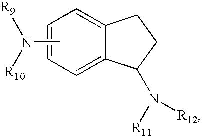
wherein R₉ and R₁₀ are both methyl and R₁₁ and R₁₂ are both H, comprising the steps of:
(a) reacting 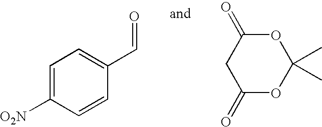
in the presence of triethylammonium formate reagent to produce 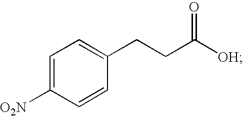
(b) reducing the product of step (a) with Pd/C in the presence of triethyl amine and formic acid to produce 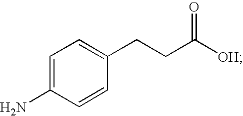
(c) reacting the product of step (b) with acetic anhydride to produce 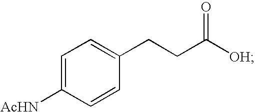
(d) reacting the product of step (c) with a cyclization agent to produce 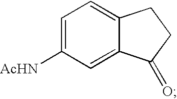
(e) removing the acetyl group of the product of step (d) by reacting it with an acid to produce 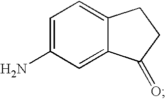
(f) reacting the product of step (e) with paraformaldehyde and hydrogen over a palladium catalyst to produce 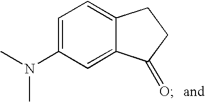
(g) reacting the product of step (f) with sodium cyanoborohydride and ammonium acetate in the presence of solvent to produce

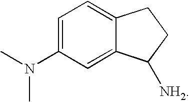

In an embodiment of this process described immediately above, the cyclization agent in step (d) is AlCl₃, with or without NaCl. In another embodiment of this process, the acid in step (e) is HCl. In a further embodiment of the immediately aforementioned process, step (g) comprises the steps of:
(1) reacting the product of step (f) with hydroxylamine to produce 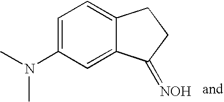
(2) reducing the product of step (1) with a reducing agent to produce

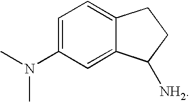

In yet a further embodiment of this process, the reducing agent in step (2) is hydrogen and a palladium catalyst.

The subject invention also provides a process for manufacturing a compound having the structure: 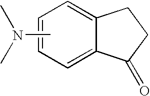
comprising reacting a compound having the structure 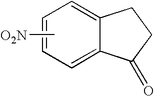
with paraformaldehyde and hydrogen over a palladium catalyst to produce the compound.

In an embodiment of this process, the compound has the structure 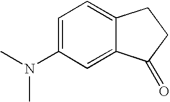
and the compound is produced by reacting a compound having the structure 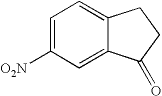
with paraformaldehyde and hydrogen over a palladium catalyst. In another embodiment of the process described immediately above, the compound has the structure 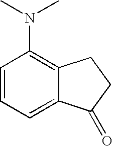
and the compound is produced by reacting a compound having the structure 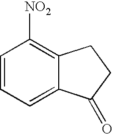
with paraformaldehyde and hydrogen over a palladium catalyst.

The subject invention also provides a compound having the structure:

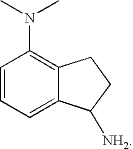

The subject invention further provides a process for manufacturing a compound having the structure: 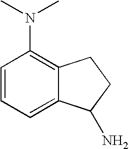
comprising:
(1) reacting a compound having the structure 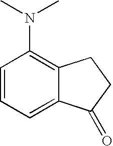
with sodium cyanoborohydride and ammonium acetate in the presence of solvent to produce

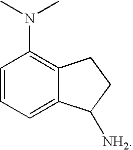

Another process for manufacturing a compound having the structure: 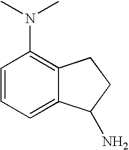
comprises:
(1) reacting a compound having the structure 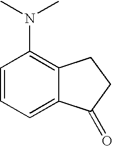
with hydroxylamine to produce 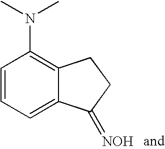
(2) reducing the product of step (1) with a reducing agent to produce

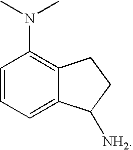

In an embodiment of this process immediately described above, the reducing agent in step (2) is hydrogen and a palladium catalyst.

The subject invention further provides a use of any one of the compounds disclosed for manufacturing a medicament useful for treating a subject suffering from a neurologic disorder or an autoimmune disorder. In one embodiment, the subject suffers from a neurologic disorder. The neurologic disorder may be any one of Alzheimer's disease, Parkinson's disease, amyotrophic lateral sclerosis or age related macular degeneration. In another embodiment, the subject suffers from an autoimmune disorder. The autoimmune disorder may be multiple sclerosis.

The subject invention also provides a use of any one of the disclosed compounds for manufacturing a medicament useful for treating a subject afflicted with an inflammatory disorder caused by the presence of reactive oxidative species. In one embodiment, the inflammatory disorder is caused by the presence of peroxynitrite in the subject. The inflammatory disorder may be any one of an inflammatory bowel disease, or rheumatoid arthritis.

The subject invention also provides a use of any one of the disclosed compounds for manufacturing a medicament useful for preventing the oxidation of lipids, proteins or deoxyribonucleic acid in a cell.

Finally, the subject invention provides a use of any one of the disclosed compounds for manufacturing a medicament useful for preventing lysis of human red blood cells by O₂ radicals.

Those skilled in the art will be familiar with the fact that some compounds of the formula (I) can exist as tautomers. The compounds of the formula (I) are therefore also to be understood as meaning herein the relevant tautomers, even when not mentioned specifically in each individual case. This invention also relates to the use of all such tautomers and mixtures thereof.

It will be noted that the structure of some of the compounds of this invention includes asymmetric carbon atoms and thus occur as racemates and racemic mixtures, single enantiomers, diastereomeric mixtures and individual diastereomers. All such isomeric forms of these compounds are expressly included in this invention. Each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis.

As set out above, certain embodiments of the present compounds can contain a basic functional group, such as amino or alkylamino, and are thus capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The term “pharmaceutically acceptable salts” as used herein also includes a quaternary ammonium salt.

When the compounds of the present invention are administered as pharmaceuticals, to humans and mammals, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, pills, tablets, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert dilutents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert dilutents, the oral compositions can also include adjuvants such as wetting agents, emulsifying and suspending agents, sweetening, flavoring, coloring, perfuming and preservative agents.

Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal,intraperitoneal, transtracheal, subcutaneous, subcuticular, intraarticular, subcapsular, subarachnoid, intraspinal and intrasternal injection and infusion.

The phrases “systemic administration,” “administered systematically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above.

If desired, the effective daily dose of the active compound may be administered as two, three, four, five, six or more sub-doses administered separately at appropriate intervals throughout the day, optionally, in unit dosage forms.

In one embodiment, the dosage of the active compound administered is 50-500 mg per day.

The phrase “neurologic disorder” as used herein refers to a disorder whose adverse affects are localized in the nervous system. For instance, neurologic disorder as used herein may refer to secondary degeneration which may otherwise follow primary nervous system (NS) injury, e.g., closed head injuries and blunt trauma, such as those caused by participation in dangerous sports, penetrating trauma, such as gunshot wounds, hemorrhagic stroke, ischemic stroke, glaucoma, cerebral ischemia, or damages caused by surgery such as tumor excision; degenerative process,. e.g., degeneration occurring in either gray or white matter (or both) as a result of various diseases or disorders, including, without limitation: diabetic neuropathy, senile dementias, Alzheimer's disease, Parkinson's Disease, facial nerve (Bell's) palsy, glaucoma, Huntington's chorea, amyotrophic lateral sclerosis (ALS), status epilepticus, non-arteritic optic neuropathy, intervertebral disc herniation, vitamin deficiency, prion diseases such as Creutzfeldt-Jakob disease, carpal. tunnel syndrome, peripheral neuropathies associated with various diseases, including but not limited to, uremia, porphyria, hypoglycemia, Sjorgren Larsson syndrome, acute sensory neuropathy, chronic ataxic neuropathy, biliary cirrhosis, primary amyloidosis, obstructive lung diseases, acromegaly, malabsorption syndromes, polycythemia vera, IgA and IgG gammapathies, complications of various drugs (e.g., metronidazole) and toxins (e.g., alcohol or organophosphates), Charcot-Marie-Tooth disease, ataxia telangectasia, Friedreich's ataxia, amyloid polyneuropathies, adrenomyeloneuropathy, Giant axonal neuropathy, Refsum's disease, Fabry's disease, lipoproteinemia, etc; and other clinical conditions including epilepsy, amnesia, anxiety, hyperalgesia, psychosis, seizures, abnormally elevated intraocular pressure, oxidative stress, and opiate tolerance and dependence.

While it is possible for a compound of the present invention to be administered alone, it is preferable to administer the compound as a pharmaceutical composition.

This invention will be better understood from the Experimental Details which follow. However, one skilled in the art will readily appreciate that the specific methods and results discussed are merely illustrative of the invention as described more fully in the claims which follow thereafter.

Experimental Details

Dimethylamino indanones serve as precursors to dimethylamino aminoindans, and were previously prepared by reductive methylation (paraformaldehyde, hydrogen) of nitro indanones, as described in Hasbun et al, J. Med. Chem., (1973) 16: 847 and Biggs et al, J. Med. Chem., (1976) 19: 472. Nitration of 1-indanone affords a mixture of two regioisomers (4- and 6-nitro indanone) which may be separated by column chromatography. The compounds of the present invention were prepared as illustrated in the synthesis schemes summarized below.

Dimethylamino indanones were prepared by reductive methylation (paraformaldehyde, hydrogen) of either nitroindanones or aminoindanones (Scheme 1). Preparation of aminoindanones was either by the reduction of nitroindanones, or by hydrolysis of N-acetyl aminoindanones (Scheme 1).

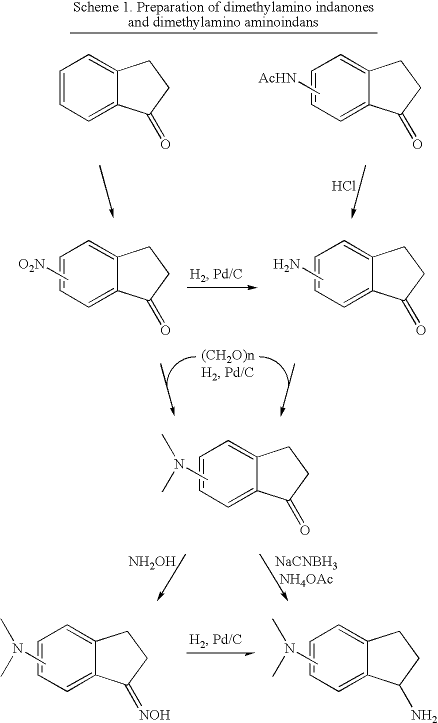

The latter are obtained by reacting nitrobenzaldehydes with Meldrum's acid, converting the nitro group to an acylamino group and cyclyzing to an indanone structure (Scheme 2). This approach represents a useful entry for the synthesis of pure regioisomers, obviating the need for column chromatography.

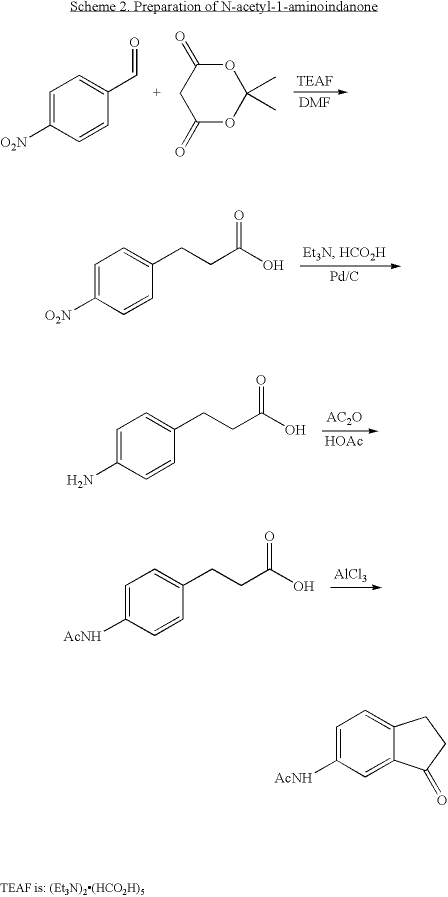

Preparation of dimethylamino aminoindans was by reductive amination of the carbonyl moiety of dimethylamino indanones either by a two-step process via the oxime, or in one step with ammonium acetate and sodium cyanoborohydride (Scheme 1).

Compounds of general formula I bearing the indanyl moiety at the N1 position may be prepared by reacting indanyl urea with ethyl cyanoacetate. The corresponding 5-amino analogues are prepared by nitrosation at position 5, followed by reduction of the nitroso group (Scheme 3).

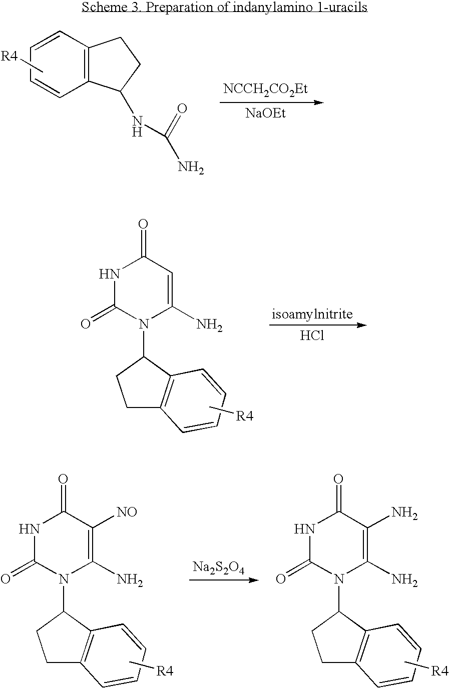

Compounds of general formula I bearing the indanyl moiety at the N6 position may be prepared by reacting 1-aminoindans with uracil derivatives having either chloro or amino groups at the 6 position, in a high boiling solvent such as DMSO. The corresponding 5-amino analogues are prepared as described above (Scheme 4).

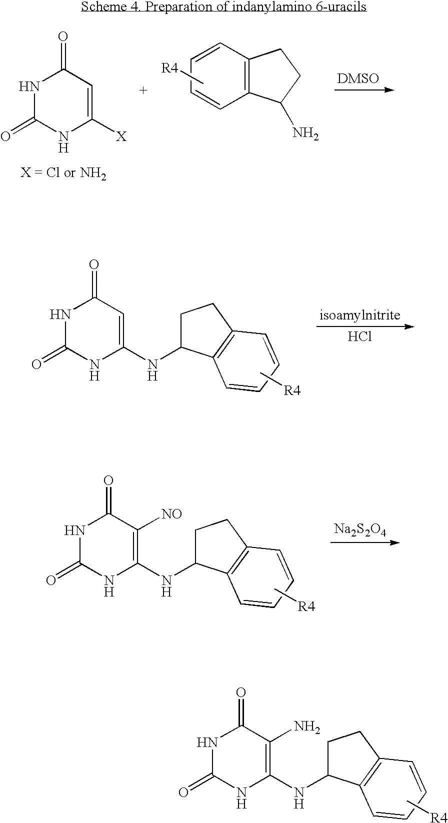

### EXAMPLE 1

### 6-Dimethylamino-1-aminoindan

### 1.1 From 6-Dimethylamino-1-indanone

### 1.1.1 6-Dimethylamino-1-indanone

6-nitro-1-indanone (6.67 g, 0.038 mol) was reductively methylated as described by Hasbun et al., J. Med. Chem. (1973), 16:847 and Biggs et al., J. Med. Chem. (1976) 19:472. This crude product was purified either by crystallization (75 ml, 1:2 iPrOH:H₂O), in which case 4.88 g (74%) yellow crystalline solid (mp: 78-80° C.) was obtained, or by column chromatography (hexane: EtOAc-2:1).

¹H-NMR (CDCl₃) δ: 7.33 (d, 1H, J=8 Hz, H-4), 7.05 (dd, 1H, J=8, 2.5 Hz, H-5), 7.00 (d, 1H, J=2.5 Hz, H-7), 3.01 (t, 2 H, J=6 Hz, H-2,3) , 2.97 (s, 6 H, NMe₂), 2.66 (t, 2H, J=6 Hz, H-2,3). ¹³C (CDCl₃) δ: 207.78 (C=O), 150.11, 143.60, 137.71 (3×C, C-3a, C-7a, C-6), 126.71, 120.52, 105.11 (3×CH, C-4, C-5, C-7), 40.78 (NMe₂), 36.89 (CH₂CO), 24.69 (COCH₂CH₂). MS (CI) (NH₃) m/z (176, MH⁺).

### 1.1.2. 6-Dimethylamino-1-aminoindan

### 1.1.2.1 Via the Oxime

A mixture of 6-dimethylamino-indanone (10 g, 0.057 mol), hydroxylamine.HCl (6 g, 0.086 mol), sodium acetate (7.7 g, 0.094 mol) in EtOH (40 ml) and water (40 ml) was refluxed for 2 h. After cooling to rt, water (50 ml) was added. The solid was collected by filtration, washed thoroughly with water and dried to give 6-dimethylamino-indanone oxime as a yellow powder (8 g, 74%).

¹H-NMR (CDCl₃) δ: 7.18 (d, 1H, J=8 Hz, H-4), 7.06 (d, 1H, J=2 Hz, H-7), 6.85 (dd, 1H, J=8, 2 Hz, H-5), 2.95 and 2.96 (2×s, 10 H, NMe₂ and CH₂CH₂). ¹³C (CDCl₃) δ: 164.81 (C═NOH), 150.07, 137.38, 136.64 (3×C, C-3a, C-7a, C-6), 125.80, 116.80, 104.59 (3×CH, C-4, C-5, C-7), 41.91 (NMe₂), 27.55, 26.61 (CH₂CH₂). MS (CI) (NH₃) m/z (191, MH⁺), (175, MH⁺—O).

The oxime (8 g, 0.042 mol) was dissolved in AcOH (90 ml) and H₂SO₄ conc. (0.088 mol, 8.66 g, 4.7 ml) was added, followed by Pd/C (0.8 g). The mixture was hydrogenated (70 psi) at 50° C. for 3 h, filtered, 0.8 g of fresh Pd/C catalyst was added and the mixture further hydrogenated for additional 3 h. The reaction mixture was cooled to rt, filtered (celite) and the filtrate evaporated to dryness to give a dark oil. Water (100 ml) was added and the pH of the solution was adjusted by solid NaOH to a pH range of 10-11. The basic solution was extracted with toluene (3×100 ml), followed by dichloromethane (2×100 ml). The organic phases were combined, dried (MgSO₄), filtered and evaporated to dryness to give 6.5 g crude liquid dark product. The latter was distilled as described in Ex. 2

### 1.1.2.2 Via Reductive Amination

6-Dimethylamino-1-indanone (6.15 g, 0.035 mol), dry NH₄OAc (30 g, 0.389 mol), and NaCNBH₃ (3.688 g, 0.059 mol) were dissolved in MeOH (200 ml) and the reaction mixture refluxed under N₂ for 6 h, then stirred under N₂ for 20 h at rt. MeOH was removed by evaporation to give an oily liquid which was partitioned between water (100 ml) and dichloromethane (500 ml). The aqueous phase was made basic by adding 4N KOH solution until reaching a pH of 10. After a few minutes of vigorous stirring the two layers were separated, and the water phase was extracted with dichloromethane (3×100 ml). The combined organic phase was washed with water (100 ml), dried (MgSO₄) and filtered. Evaporation of the filtrate gave a brown oily liquid (6 g), which was purified by column chromatography (dichloromethane: MeOH:NH₄OH-95:5:1), to afford 3.72 g (61%) of 6-Dimethylamino-1-aminoindan as a yellowish oil.

¹H-NMR (CDCl₃) δ: 7.08 (d, 1H, J=8 Hz, H-4), 6.77 (d, 1H, J=2 Hz, H-7), 6.64 (dd, 1H, J=8, 2 Hz, H-5), 4.28 (t, 1H, J=8 Hz, CH—NH₂), 2.92 (s, 6H, NMe₂), 2.83 (ddd, 1H, J=3, 7, 15 Hz, CHCH₂CH₂); 2.71 (dt, 1H, J=15, 8 Hz, CHCH₂CH₂), 2.50 (dddd, 1H, J=15, 8, 7, 3, Hz, CHCH₂CH₂),1.88 (br s, 2H, NH₂), 1.67 (dq, 1H, J=15, 8 Hz, CHCH₂CH₂); ¹³C (CDCl₃) δ 150.28, 148.16, 131.31 (3×C, C-3a, C-7a, C-6), 124.89, 113.04, 107.89 (3×CH, C-4, C-5, C-7), 57.56 (CH—NH2) 41.24 (NMe₂), 37.89 (CHCH₂CH₂), 29.28 (CHCH₂CH₂). MS (CI) (NH₃) m/z (176, MH⁺), (159, MH⁺—NH₃).

### 1.2 From 6-Amino-1-indanone

### 1.2.1 3-(4-Nitrophenyl) propionic acid

A mixture of DMF (400 ml), 4-nitro-benzaldehyde (302.2 g, 2.0 mol), Meldrum's acid (288.3 g, 2.0 mol) and triethylammonium formate reagent (TEAF; (Et₃N)₂.(HCOOH)₅) (500 ml, prepared by adding 336 ml of triethylamine dropwise to 228 ml of cooled and stirred formic acid) was stirred at 45-50° C. for 7 h then set aside for 14 h at rt. The resultant solution was heated at 100° C. for 2.5 h, cooled to rt and poured onto a mixture of ice-water (2400 g) and conc. HCl (240 ml). The solid was collected by filtration, washed with water and dried (375.3 g, 96.1%). The crude product was crystallized from ethanol:water to give 292.8 g (75%), mp: 164-166° C.

### 1.2.2 3-(4-Aminophenyl) propionic acid

Formic acid (62.3 ml, 1.65 mol) was added dropwise over a period of 2 h to a well stirred and heated (90-95° C.) mixture of triethylamine (301 ml), 3-(4-nitrophenyl) propionic acid (97.6 g, 0.5 mol) and 5% palladium on charcoal (4.26 g). The reaction mixture was refluxed for 1.5 h, and water (500 ml) and charcoal were added and the mixture was stirred for 0.5 h. Charcoal was filtered off, washed with water, and the combined filtrates were evaporated to a volume of 400 ml under reduced pressure, and the pH adjusted to 5 with conc. HCl. The suspension was cooled overnight, the solid was collected by filtration, washed with water and dried to give 64.0 g (77.5%), mp: 129-132° C.

### 1.2.3 3-(4-Acetylaminophenyl) propionic acid

3-(4-Aminophenyl) propionic acid (165.2 g, 1 mol) was added to acetic acid (130 ml), and acetic anhydride (99.1 ml, 1.05 mol) was added dropwise. The reaction mixture was gently refluxed for 0.5 h, left to cool to 90° C., and poured onto crushed ice (1 kg). The solid was collected by filtration, washed with water and crystallized from 50% ethanol to give 155.4 g (75%),

mp: 139-141° C. The mother liquor was concentrated under reduced pressure and chloroform was added. The two-phase mixture was thoroughly shaken, cooled overnight, and filtered. A second crop was thus obtained (20.7 g, 10%) mp: 137-140° C. Mp of an analytical sample (recrystallized from 50% EtOH) was 141-143° C.

### 1.2.4 6-Acetylaminoindanone

A mixture of 3-(4-acetylaminophenyl) propionic acid (15 g) and sodium chloride (48 g) was placed in a vessel immersed in an oil bath at 50° C. Aluminium chloride (240 g) was added and the mixture heated at 140° C. for about 0.5 h. After HCl evolution subsided, an additional amount (47.2 g) of 3-(4-acetylaminophenyl) propionic acid was added portionwise over 20 min. After completion of addition, the mixture was heated at 140° C. for about 10 min. A mixture of AlCl₃ (40 g) and NaCl (8 g) was added and the reaction mixture was heated at 140° C. for 40 min. The hot dark melt was poured onto a mixture of ice (1.5 kg) and conc. HCl (60 ml). The solid was removed by filtration and washed with water. The combined filtrates were extracted with CHCl₃, and the organic phase was treated with charcoal, dried and evaporated to dryness under reduced pressure to give 48.3 g (85%), mp: 176-180° C. Mp of an analytical sample (recrystallized from EtOH) was 180-182° C.

### 1.2.5 6-Aminoindanone

A mixture of 6-acetylamino-1-indanone (75.7 g, 0.4 mol) and 2 N HCl (800 ml) was refluxed for 1 h. The brown solution was treated with charcoal and cooled to rt. Ice was added to the solution, and the pH was adjusted to 9 with 20% sodium hydroxide solution. The solid was collected by filtration, washed with water and dried. The crude product (54.5 g, 92.6%) was crystallized from acetone to give 42.4 g (72%), mp: 173-175° C. and a second crop (7.6 g, 13%, mp: 168-71° C).

### 1.2.6 6-Dimethylamino-1-indanone

A mixture of 6-amino indanone (5 g, 34 mmol), paraformaldehyde (8 g, 260 mmol) and Pd/C (10%, 54% water, 0.7 g) was stirred in MeOH(100 ml) and hydrogenated with hydrogen (70 psi) at 50 C for 4 h. The reaction mixture was filtered through celite and the filtrate evaporated to dryness to give an oily liquid which was crystallized from an isopropanol/water mixture as yellow crystals (4.55 g, 76%).

### EXAMPLE 2

### 4-Dimethylamino-1-aminoindan

### 2.1 4-dimethylamino indanone

4-Nitro-1-indanone (9.39 g, 0.053 mol) was hydrogenated in MeOH (150 ml) with paraformaldehyde (13.72 g, 0.457 mol) and 5% Pd/C (1.125 g, 54.2% water) as described for the 6-isomer in Example 1. Following the work-up and purification procedures employed for the 6-isomer in Example 1, 6.35 g (69%) of 4-dimethylamino indanone was obtained.

¹H-NMR (CDCl₃) δ: 7.35 (dd, 1H, J=7.5 Hz, H-5), 7.33 (t, 1H, J=7.5 Hz, H-6), 7.08 (dd, 1H, J=7.5, 1 Hz, H-7), 3.13 (t, 2 H, J=6 Hz, H-2,3), 2.88 (s, 6 H, NMe₂), 2.68 (m, 2H, H-2,3). MS (CI) (NH₃) m/z (176, MH⁺).

### 2.2 4-dimethylamino-1-aminoindan

4-Dimethylamino indanone (8.6 g, 0.049 mol), NH₄OAc (30 g, 0.389 mol) and NaCNBH₄ (5.15 g, 0.082 mol) were dissolved in MeOH (250 ml) and the reaction mixture was refluxed under N₂ for 8 h and then stirred for 15 h at rt. The reaction mixture was worked up as described in Example 1, to afford 8.4 g of the crude product as a brown liquid. The latter was distilled (100-105° C./0.2 mmHg) to give 4.0 g (46%) of the title compound as a clear liquid.

¹H-NMR (CDCl₃) δ: 7.16 (t, 1H, J=8 Hz, H-6), 6.93 (d, 1H, J=8 Hz, H-5), 6.75 (d, 1H, J=8 Hz, H-7), 4.32 (t, 1H, J=8 Hz, CH—NH₂), 2.97 (ddd, 1H, J=3, 8, 15 Hz, CHCH₂CH₂), 2.78 (s, 6H, NMe₂ and m, 1H, CHCH₂CH₂), 2.50 (dddd, 1H, J=15, 8, 7, 3 Hz, CHCH₂CH₂), 1.66 (dq, 1H, J=12, 8 Hz, CHCH₂CH₂); ¹³C (CDCl₃) δ 149.86, 149.12, 133.39 (3×C, C-3a, C-7a, C-6), 127.53, 115.92, 114.80 (3×CH, C-4, C-5, C-7), 57.38 (CH—NH₂) 42.86 (NMe₂), 37.48 (CHCH₂CH₂), 29.65 (CHCH₂CH₂). MS (CI) (NH₃) m/z (176, MH⁺), (159, MH⁺—NH₃).

### EXAMPLE 3

### 6-amino-1-indan-1-yl-1H-pyrimidine-2,4-dione (1)

1-Indanylurea (57.23 g, 0.325 mol, prepared from 1-aminoindan and sodium cyanate) and ethyl cyanoacetate (37.65 g, 0.33 mol) were added to a solution of sodium (8.23 g, 0.36 g atom) in ethanol (420 ml). A clear solution was obtained on stirring and heating under reflux. The solution was maintained at reflux for 24 h, cooled to 40° C. and treated with 1 N HCl (500 ml). The precipitated solid (unchanged urea, 14.74 g, 25.8% recovery) was removed by filtration and the filtrate (including washings) was ice-cooled and the solid collected, washed with ethanol/water 2:3 and water and dried in vacuo (37.3 g, 47.2% yield, 63.6% based on net urea consumed). A sample of the product (5.11 g) was recrystallised from acetic acid (45 ml) and water (35 ml) using decolourising charcoal to give the product as a hemiacetate (2.32 g), mp >295° C. C₁₃H₁₃N₃O₂. 0.5 C₂H₄O requires: C, 61.53; H, 5.53; N, 15.38%; Found: C, 61.62; H, 5.59; N, 15.57%. MS (CI/NH3): 244 (MH⁺, 100%).

¹H NMR (DMSO-d₆) δ ppm: 11.97 (s, 0.5H, MeCOOH) 10.55 and 10.15 (v br s, H), 7.18 (br m) and 6.97 (br s) (6H, 4 Ar—H and NH₂); 5.65 (br s) and 5.55 (br s) (H, C1-H); 4.625 (s, H, C5-H); 3.05-3.2 (m, H, C3-H); 2.8-2.95 (m, H, C3-H); 2.25-2.45 (m, 2H, C2-H₂); 1.90 (S, 1.5H, 0.5 CH₃COOH) . IR: 3459, 3321, 3247, 1697, 1636, 1578, 1490, 1387, 1289, 821, 770, 783 cm⁻.

### EXAMPLE 4

### 6-amino-1-(6-methoxy-indan-1-yl)-1H-pyrimidine-2,4-dione (2)

### 4.1 (6-Methoxy-indan-1-yl)-urea

A mixture of 6-methoxy-1-indanamine (16.26 g, 0.10 mol), a solution of 10.2 N HCl (9.3 ml, 0.09 mol) and water (85 ml) was heated to 60° C. and treated with sodium cyanate (6.79 g, 0.10 mol) in portions. After 2 h the mixture was cooled, diluted with a little water and filtered. The crude product was crystallized from ethanol (250 ml) to give a white solid (15.3 g, 74.5%), mp 213° C. MS (CI/NH3): 207 (MH⁺, 100%), 147 (5%, MH⁺—CO(NH₂)₂)

¹H NMR (DMSO-d₆) δ ppm: 7.11 (d, J=9 Hz, H, C4-H); 6.76 (m, 2H, C7-H, C5-H); 6.31 (d, J=8.4 Hz, H, NH); 5.50 (s, 2H, NH₂); 5.01 (q, J=7.8 Hz, H, C1-H); 3.71 (s, 3H, MeO); 2.73-2.86 (m, H, C3-H); 2.58-2.73 (m, H, C3-H); 2.3-2.43 (m, H, C2-H); 1.58-1.74 (m, H, C2-H).

IR: 3413, 3330, 3207, 1649, 1593, 1287, 1173, 1026, 817 cm⁻.

4.2 6-amino-1-(6-methoxy-indan-1-yl)-1H-pyrimidine-2,4-dione (6-Methoxy-indan-1-yl)-urea (10.02 g, 0.049 mol) and ethyl cyanoacetate (5.41 g, 0.048 mol) were added to a solution of sodium (1.2 g, 0.05 g atom) in ethanol (65 ml). A clear solution was obtained on stirring. The solution was maintained for 25 h at reflux, cooled to 40° C. and treated with a solution of 1N HCl (80 ml). The brown mixture was cooled to 20° C., and the solid was collected by filtration, washed with ethanol/water and dried in vacuo at 60° C. (8.47 g). This crude product (containing some starting material) was purified by column chromatography (ethyl acetate/methanol 97:3 v/v) to give the title compound (3.5 g, 26.2%) as a brown solid. MS (CI/NH3) (IAU203A): 274 (MH⁺).

¹H NMR (DMSO-d₆) δ ppm: 10.62, 10.18 (br s,s, N3-H); 6.3-7.3 (br mm, 5H, Ar—H₃ and NH₂); 5.64, 5.53 (br tr, s, H, C1-H); 4.64 (s, H, C5-H); 3.71, 3.69 (s,s, 3H, MeO); 2.95-3.09 (m, H, C3-H); 2.6-2.95 (m, H, C3-H); 2.2-2.47 (m, 2H, C2-H).

IR: 3455, 3332, 1700, 1637, 1578, 1490, 1205 cm⁻.

### EXAMPLE 5

### 5,6-diamino-1-indan-1-yl-1H-pyrimidine-2,4-dione (3)

5.1 5-Nitroso-6-amino-1-indan-1-yl-1H-pyrimidine-2,4-dione 6-Amino-1-indan-1-yl-1H-pyrimidine-2,4-dione (12.34 g, 0.051 mol) stirred in ethanol (340 ml) was treated with isoamyl nitrite (13.8 ml, 0.1 mole) followed by conc. HCl (1.2 ml). The violet solid which began to precipitate was collected after 6 h stirring, washed with ethanol and ether and dried in vacuo at 60° C. (7.93 g, 57.4%). A sample (0.38 g) was crystallized from a methanol/water mixture to give a purple solid (0.26 g) , mp >260° C. MS (CI/NH3) : 273 (100%, MH⁺).

5.2 5,6-Diamino-1-indan-1-yl-1H-pyrimidine-2,4-dione Sodium dithionite monohydrate (10.62 g, 0.055 mol) was added over 10 min to a stirred slurry of 5-nitroso-6-amino-1-indan-1-yl-1H-pyrimidine-2,4-dione (5.12 g, 0.019 mol) in water (78 ml) at 65° C. After 1 h, the greenish mixture was ice cooled, filtered and the solid washed well with water, ethanol and ether (3.29 g, 67.7%). It was heated with ethanol (80 ml), treated alternately with water and more ethanol at reflux, and the pale brown solution treated with active charcoal. Hot filtration and prolonged cooling resulted in a pale yellow solid which was dried overnight in vacuo at 60° C. (2.11 g, 43.4%), mp 218° C. (dec).

C₁₃H₁₄N₄O₂ requires: C, 60.45; H, 5.46; N, 21.69%. Found: C, 60.11; H, 5.48; N, 21.08%.

¹H NMR (DMSO-d₆) δ ppm: 10.42 (br s, H, N3-H); 7.19 and 7.0 (2×s, 4H, Ar—H₄); 6.67, 6.30 (2×s, 2H, C6-NH₂); 5.72, 4.76 (2×v br s, H, C1-H); 3.12 (m, H, C3-H); 2.88 (s, 3H, C3-H, NH₂); 2.41 (m., 2H, C2-H₂). MS (CI/NH3): 259 (MH⁺, 100%).

IR: 3482, 3368, 2968, 1701, 1625, 1408, 1255, 755 cm⁻.

### EXAMPLE 6

### 5-amino-6-(indan-1-ylamino)-1H-pyrimidine-2,4-dione (4)

### 6.1 5-Nitroso-6-(indan-1-ylamino)-1H-pyrimidine-2,4-dione

6-(Indan-1-ylamino)-1H-pyrimidine-2,4-dione (Compound 5, Example 7) (12.4 g, 0.051 mol), was stirred at rt with ethanol (330 ml), treated with isoamyl nitrite (13.5 ml, 0.13 mol) and then with conc. HCl (1.2 ml). The solid which precipitated was collected after 6 h, washed with ethanol and ether and dried in vacuo (12.2 g, 87.9%). A sample (0.65 g) was recrystallized from a mixture of acetic acid (20 ml) water (15 ml) and finally a little ethanol (5 ml) to give a deep blue solid (0.39 g), mp 244° C. (dec). MS (CI/NH3): 273 (MH⁺, 70%), 255 (MH—H₂O, 100%).

C₁₃H₁₂N₄O₃ requires: C, 57.35; H, 4.44; N, 20.58% . Found: C, 56.57; H, 4.58; N, 20.10%.

¹H NMR (DMSO-d₆) δ ppm: 11.45-12.55 (v br, 0.5 H, NH); 11.34 (s, H, N3-H), 7.34 (m, 4.5H, 4 Ar—H and NH); 5.55 (q, J=7.2 Hz, H, C1-H); 3.1-3.8 (v br, 2H, NH); 2.93-3.1 (m, H, C1-H); 2.75-2.93 (m, H, C3-H); 2.58-2.72 (m, H, C2-H); 1.87-2.05 (m, H, C2-H).

IR: 3014, 2831, 1728, 1675, 1528, 1429, 1282, 1175, 794, 766 cm⁻.

### 6.2 5-Amino-6-(indan-1-ylamino)-1H-pyrimidine-2,4-dione

Crude 5-Nitroso-6-(indan-1-ylamino)-1H-pyrimidine-2,4-dione (7.16 g, 0.026 mol) was crushed, suspended with stirring in water (140 ml) and heated to 60-70° C. Sodium dithionite monohydrate (14.36 g, 0.075 mol) was added in portions over 15 min. After further stirring (1.25 h) the cooled mixture was filtered and the grey-pink solid washed with water, ethanol and ether and dried (4.9 g, 71.1%). This material was recrystallized twice from ethanol aided by water. After final freezer-cooling, the solid was collected by filtration, washed with ethanol and ether and dried in vacuo at 60° C. overnight, mp>150° C.

C₁₃H₁₄N₄O₂. 0.5 C₂H₅OH. requires: C, 59.75; H, 6.05; N, 19.93%. Found: C, 59.76; H, 6.18; N, 20.51%. MS (CI/NH3): 259 (MH⁺, 100%), 143 (MH-indene, 10%).

¹H NMR (DMSO-d₆) δ ppm: 10.45 (br s, H, N3-H); 7.24 (m, 4H, Ar—H₄); 5.86 (br d, J=9 Hz, H, C6-NH); 5.34 (br q, J=8.5, H, C1-H); 4.37 (br t, J≈5.5 Hz, 0.5H, 0.5 EtOH); 3.44 (dq, H, 0.5MeCH₂OH); 2.99-3.02 (m, H, C3-H); 2.68-2.84 (m, H, C3-H); 2.41-2.54 (m, H, C2-H); 1.76-1.91 (m., H, C2-H).

IR: 2968, 1708, 1671 (vs), 1412, 1383, 1225, 800, 749 cm⁻.

### EXAMPLE 7

### 6-(indan-1-ylamino)-1H-pyrimidine-2,4-dione (5)

1-Aminoindan hydrochloride (11.07 g, 0.069 mol) and 6-aminouracil (9.32 g, 0.073 mol) were mixed mechanically with 1-aminoindan (10.39 g, 0.78 mol) and heated to 160° C. for 5 h. The liquid mixture was allowed to cool to 140° C. While stirring vigorously and heating, ethanol (130 ml) was cautiously added through the condenser, thus obtaining a smooth thick suspension which was cooled and filtered. All remaining hard material was crushed. The solid was washed with ethanol and stirred vigorously in 2N NaOH (70 ml). After ca 3 minutes the suspension was filtered, washed with a little 2N NaOH and finally water. The solid was dried in vacuo at 60° C. overnight (15.9 g). It was crystallized by dissolution in acetic acid (125 ml), treatment with charcoal, and after hot filtration, treatment at reflux with water (40 ml). After cooling, the solid was collected, washed with dilute acetic acid, ethanol and ether and dried in vacuo for 8 h (11.39 g, 63.8%). A sample was recrystallized as follows: It was taken up (4.9 g) in refluxing acetic acid (60 ml), filtered hot through hiflo and the refluxing mixture treated with water (35 ml). The solid obtained on gradual cooling was collected, washed and dried at 60° C. in vacuo (3.95 g), mp 271° C.

C₁₃H₁₃N₃O₂ requires: C, 64.19; H, 5.39; N, 17.27%. Found: C, 63.92; H, 5.58; N, 17.57%.

MS (CI/NH3): 244 (MH⁺, 100%).

¹H NMR (DMSO-d₆) δ ppm: 10.25 (br s, N3-H); 9.68 (br s, N1-H), 7.3 (m, 4H, Ar—H₄); 6.44 (d, J=7.5 Hz, H, IndNH); 4.94 (q, J=7.2 Hz, H, C1′-H); 4.66 (d, J=1.5 Hz, H, C5-H); 2.89-3.02 (m, H, C3′-H); 2.75-2.89 (m, H, C3′-H); 2.44-2.57 (m, H, C2′-H); 1.74-1.91 (m, H, C2′-H). IR: 3231, 1717, 1610 br, 1380, 763, 545 cm⁻.

### EXAMPLE 8

### 6-(4-dimethylamino-indan-1-ylamino)-1H-pyrimidine-2,4-dione HCl (6)

4-Dimethylamino-1-aminoindan (8.5 g, 48.2 mmol) and 6-chlorouracil (3.55 g, 24.2 mmol) were dissolved in DMSO (8.5 ml), heated to 115° C. and stirred for 4 h. The reaction mixture was cooled to 80° C. and glyme (ethylene glycol dimethylether) was added to provide a thick mixture which was further refluxed for 1 h. The mixture was cooled to room temperature, and the solid collected by filtration and washed well with glyme. The white solid was treated with water (50 ml) and the suspension was refluxed for 30 min, cooled to room temperature, filtered and washed thoroughly with Et₂O. A white solid (4 g, 58%) was provided. The free base was converted to the HCl salt by dissolving it in EtOH (32 ml) and HCl/EtOH (28% solution, 2.5 ml) and adding Et₂O (70 ml). The salt was collected by filtration, washed with Et₂O and dried to give an off-white powder (4.8 g, 58%). Mp 192-193° C.

¹H-NMR (free base) DMSO-d₆ δ: 10.24 (br s, 1H, CONHCO), 9.68 (br s, 1H, CONHC), 7.15 (br t, 1H, J=8 Hz, Ar), 6.85 and 6.78 (two br d, 2H, Ar), 6.42 (br d, 1H, J=6 Hz, CHNH), 4.87 (br q, 1H, J=7 Hz, CHNH), 4.64 (br s, 1H, CCHCO), 2.75 -2.90 (br s & br m, 8H, Me₂N & CH₂CH₂C), 2.45 and 1.76 (m, 2H, CHCH₂);

¹³C (free base) DMSO-d₆ δ: 164.27 (CHCO), 153.70 (NHCNH), 150.72 (NHCONH), 149.80 (Me₂NC), 144.03, 133.59, 127.61, 116.0, 115.39, 73.27 (CHCO), 56.64 (CHNH), 42.37 (Me₂N), 33.21, 29.41; Anal. (calcd for C₁₅H₁₈N₄O₂) C, 62.92; H, 6.34; N, 19.57: Found C, 62.73; H, 6.45; N, 19.20; MS (CI) (iBu) m/z (286.11, M); HRMS (CI, iBu) exact mass calcd for C₁₅H₁₈N₄O₂ 286.1429, found 286.1450.

¹H-NMR (HCl salt) DMSO-d₆ δ: 10.38 (br s, 1H, CONHCO), 10.27 (br s, 1H, CONHC), 7.64 (br d, 1H, J=8 Hz, Ar), 7.45 and 7.39 (br t & br d, 2H, Ar), 7.20 (br d, 1H, J=6 Hz, CHNH), 5.00 (br q, 1H, J=7 Hz, CHNH), 4.74 (br s, 1H, CCHCO), 3.35 (br m, 1H, CH₂CH₂C), 3.0-3.12 (br s & m, 7H, Me₂N & CH₂CH₂C), 2.57 and 1.85 (two m, 2H, CHCH₂);

¹³C (HCl salt) DMSO-d₆ δ: 164.26 (CHCO), 154.13 (NHCNH), 150.37 (Me₂NC), 145.90 (NHCONH), 140.07, 135.55, 128.65, 124.39, 119.64, 73.09 (CHCO), 56.39 (CHNH), 44.78 (Me₂N), 32.87, 28.29; MS (CI) (NH₃) m/z (287, MH⁺).

### EXAMPLE 9

### 6-(6-dimethylamino-indan-1-ylamino)-1H-pyrimidine-2,4-dione HCl (7)

The title compound was prepared from 6-dimethylamino-1-aminoindan (9.6 g, 54.5 mmol) and 6-chlorouracil (4.01 g, 27.4 mmol) according to the procedure described in Example 8. Thus, 6.2 g (70%) were obtained, mp 211-213° C.

¹H-NMR (free base) DMSO d₆ δ: 10.24 (br s, 1H, CONHCO), 9.63 (br s, 1H, CONHC), 7.10 (d, 1H, J=8 Hz, Ar), 6.68 (br d, 2H, Ar), 6.42 (br d, 1H, J=6 Hz, CHNH), 4.83 (br q, 1H, J=7 Hz, CHNH), 4.64 (br s, 1H, CCHCO), 2.86 and 2.70 (br s & br m, 8H, Me₂N & CH₂CH₂C), 2.46 and 1.75 (m, 2H);

¹H-NMR (for HCl salt) in DMSO d₆ δ: 10.36 (br s, 1H, CONHCO), 10.21 (br s, 1H, CONHC), 7.58 (br d, 2H, J=8 Hz, Ar), 7.42 (br d, 1H, Ar), 7.22 (br d, 1H, J=6 Hz, CHNH), 4.99 (br q, 1H, J=7 Hz, CHNH), 4.72 (br s, 1H, CCHCO), 3.07 (br s, 6H, Me₂N), 2.96 and 2.84 (two m, 2H, CH₂CH₂C), 2.55 and 1.82 (two m, 2H);

¹³C (free base) DMSO d₆ δ: 164.27 (CHCO), 153.64 (NHCONH), 150.70 (NHCNH), 150.08 (Me₂NC), 143.85, 130.62, 124.94, 113.22, 107.94 (Me₂NCCH), 73.23 (CHCO), 56.87 (CHNH), 40.62 (Me₂N), 33.28, 28.75;

¹³C (for HCl salt) DMSO d₆ δ: 164.37 (CHCO), 154.17 (NHCONH), 150.50 (NHCNH), 144.42 (Me₂NC), 143.34, 142.70, 126.04, 119.76, 115.63 (Me₂NCCH), 73.16 (CHCO), 56.47 (CHNH), 45.18 (Me₂N), 33.47, 29.34; MS (CI) (NH₃) m/z (287, MH⁺).

Anal. calcd for C₁₅H₁₈N₄O₂.HCl. 1.5 H₂O: C, 55.80; H, 6.81; Cl, 10.98; N, 17.34; found: C, 51.93; H, 6.28; Cl, 10.30; N, 15.94.

### EXAMPLE 10

### 6-(6-(N-methyl-N-ethyl-carbamoyloxy)-indan-1-ylamino)-1H-pyrimidine-2,4-dione (8)

6-(N-Methyl-N-ethylcarbamyloxy)-1-indanamine hydrochloride (5.4 g, 0.020 mol) in water (50 ml) was treated with 2N NaOH (20 ml, 0.04 mol), and the free base was extracted with methylene dichloride, washed, dried and the solvent evaporated (4.78 g, 100%). To this (the free base), was added the above hydrochloride (6.295 g, 0.023 mol) and 6-aminouracil (0.022 mol) and the mixture heated to 160° C. for 2 h, cooled to 140° C., ethanol (50 ml) was added during vigorous stirring and the thick suspension reheated to break up some hard material. The solid was collected, washed and stirred with 2N NaOH (20 ml, 0.04 mol), and acetic acid (3 ml, 0.05 mol) was added. The white precipitate was collected by filtration, and dissolved in boiling ethanol (200 ml) and water (50 ml). The turbid solution was filtered hot and the precipitated solid collected by filtration (1.12 g, 15%).

MS (DCI/CH₄) : 345 (25%, MH⁺) , 218 (20%, MH⁺-aminouracil) ; 117 (25%, indanyl⁺).

¹H NMR (DMSO-d₆) δ ppm: 10.26, 10.09 (s, br s, H, N3-H); 9.68, 9.28 (br,br, H, N1-H); 7.27 (d, J=7.8 Hz, H, Ind4-H); 7.03 (s) and 7.00 (dd, J=7.8, 2.1 Hz) (2H, Ind7-H and Ind5-H); 6.5 (d, J=7.4 Hz, H, IndNH); 4.95 (q, J=7.2 Hz, H, Ind1-H); 4.66 (s, H, C5-H); 3.41 (hex, H) and 3.30 (hex, H) (CH₂N rot); 3.00 and 2.89 (s,s); 2.77-3.0 (m, 5H, CH₃ rot and Ind3-H₂); 2.53 (m, H, Ind2-H); 1.83 (m, H, Ind2-H); 1.17 (tr, J=6.9 Hz) and 1.09 (tr, J=6.9 Hz) (3H, CH₃CH₂). IR: 3218, 1714, 1598, 1235, 1170, 837, 547 cm⁻.

### EXAMPLE 11

### 5-amino-6-(6-(N-methyl-N-ethyl-carbamoyloxy)-indan-1-ylamino)-1H-pyrimidine-2,4-dione (9)

### 11.1 5-Nitroso-6-(6-(N-methyl-N-ethyl-carbamoyloxy)-indan-1-ylamino)-1H-pyrimidine-2,4-dione

6-(6-(N-methyl-N-ethyl-carbamoyloxy)-indan-1-ylamino)-1H-pyrimidine-2,4-dione (0.35 g, 0.001 mol) was slurried in ethanol (6.8 ml) and treated with isoamyl nitrite (0.29 ml, 0.002 mol) and conc. HCl (1 drop). After 1.75 h the mixture was filtered through hiflo, evaporated to a purple syrup and triturated with ether. The collected solid was washed with ether and dried (0.325 g, 86%).

MSCI(CH₄): 282 (100%), 252 (10%, 282-NO); 147 (20%).

### 11.2 5-Amino-6-(6-(N-methyl-N-ethyl-carbamoyloxy)-indan-1-ylamino)-1H-pyrimidine-2,4-dione

5-Nitroso-6-(6-(N-methyl-N-ethyl-carbamoyloxy)-indan-1-ylamino)-1H-pyrimidine-2,4-dione (0.27 g, 0.0007 mol) was stirred in water (3 ml) at 65° C. and treated with sodium dithionite (0.39 g, 0.002 mol). The resultant sticky material was dissolved in ethanol, diluted with water and treated with more dithionite (0.27 g, 0.0014 mol) and combined with the original brown supernatant. This solution was treated with more dithionite (0.22 g, 0.001 mol) and kept at 60° C. for 20 min. The clear yellow solution was evaporated in vacuo to remove ethanol, and the residue triturated with water and the suspension thus obtained was cooled and filtered to give 0.06 g (23%).

MS (DCI/NH₃): 360 (50%, MH⁺), 345 (15%), 218 (100%, MH⁺-diaminouracil), 143 (55%, diaminouracil+H⁺), 128 (20%, 5-aminouracil+H⁺).

### EXAMPLE 12

### Biological Activity of Compounds of the Invention

1. Antioxidant Activity

Evaluation of Antioxidant Properties of the Compounds in vitro:

Hypochlorite (HOCl) hemolysis of human red blood cells. The reaction is based on the ability of O₂ radicals to lyse red blood cells. Antioxidant compounds such as ascorbic acid and 4-hydroxy-2,2,6,6-tetramethyl-piperidine-1-oxyl (TEMPOL) prevent membrane damage in a dose dependent manner. All compounds were dissolved at a concentration of 5 mg/ml and aliquots were tested for their ability to prevent lysis.

Chemical Chemiluminescence (without Cells)

Luminescence was generated in vials containing the following compounds: Luminol+SIN-1(generator of NO radicals)+selenite+BSA. This reaction is inhibited by scavengers of peroxynitrites, as well as scavengers of NO and O₂ free radicals.

PMA-induced oxidative burst in neutrophils: The cells were activated with PMA (10 ng/ml) for 30 min and incubated with 2,7-dichlorodihydrofluorescin diacetate (DCFH), which, in the presence of O₂ radicals and peroxynitrites, is converted to a fluorescent compound. The fluorescent cells are detected in a cell sorter (FACS).

Stastistical evaluation of the results: The raw data was incorporated into Sigma-Stat and the means±SEM of the different groups and treatments were compared using several tests suggested by the program. The results are shown in Table 1.

TABLE 1

| | HOCl | % Chemiluminescence | PMA % of cells |
|---|---|---|---|
| Compound | % lysis | inhibition | activated |
| 1 | 75 | 0 | 0 |
| 5 | 100 | 75 | 80 |
| 3 | 72 | 38 | 30 |
| 6 | 44 | 56 | NT |
| 7 | 55 | 62 | NT |
| 4-hydroxy- | 50 | 78 | 80 |
| 2,2,6,6- | | | |
| tetramethyl- | | | |
| piperidine-1- | | | |
| oxyl (TEMPOL) | | | |
| N-Hydroxy- | 52 | 42 | 60 |
| tetramethyl-4- | | | |
| piperidinol | | | |
| Tetramethyl-4- | 0 | 1.2 | 30 |
| piperidinol | | | |
| Ascorbic acid | 100 | 100 | 100 |

Activity of the compounds was compared to ascorbic acid.
NT = not tested

Redox Data

TABLE 2
Redox and radical-scavenging properties

| Compound | DPPNH | Cyclic voltametry - AW |
|---|---|---|
| 1 | 0 | ++ |
| 5 | 0 | +(+) |
| 4 | ++ | 0 |
| 3 | +++ | +++ |
| Uric acid | +++ | +++ |
| Ascorbic | NT | +++ |
| acid | | |

DPPNH is 1,1-diphenyl-2-picrylhydrazyl

Reaction with 1,1-diphenyl-2-picrylhydrazyl (DPPNH) is indicative of radical scavenging ability. The compounds were mixed with DPPNH (in equimolar amounts) and the consumption rate of DPPNH was measured. Compounds 3 and 4 reacted instantaneously, consuming 70% and 20% of DPPNH respectively (Table 2). The oxidation potential of the compounds was measured by cyclic voltametry. At pH 7.2 compound 3 exhibited strong electron donor activity (similar anodic wave) as ascorbic acid, slightly more negative (stronger) than uric acid. Compounds 1 and 5 also showed reducing action, but to a lesser extent than compound 3.

2. Experimental Allergic Encephalomyelitis (EAE)

2.1 PLP Induced EAE

Materials, Methods and Experimental Design:

Induction of Experimental Allergic Encephalomyelitis (EAE):

Female SJL mice (12 weeks) were inoculated with the encephalitogenic peptide of proteolipid protein (PLP 139-151) synthesized to a purity of 70% by Sigma (Israel). 150 μg of the peptide were emulsified in complete Freund's adjuvant (CFA) (Difco laboratories), supplemented with killed mycobacteria (5 mg/ml) and pertussis toxin (200 ng) (Sigma), given subcutaneously at day of inoculation only.

Mice were kept SPF conditions and given water and food ad libitum. Mice were daily observed for clinical signs from day 10 to day 18-21 post inoculation.

The clinical score used is as follows: 0—no clinical signs; 0.5—loss of right reflex; 1—distal limp tail; 2—complete limp tail; 3—ataxia; 4—early paralysis (one hind limb); 5—complete hind legs paralysis and moribund state; 6—death.

Treatment with Test Compounds:

Compounds were dissolved in 2-hydroxypropyl beta cyclodextrin (40% water solution). Treatment started at day 1 post inoculation. The compounds (50 mg/kg) and vehicle were given orally by gavage every day until day 20 post inoculation.

As shown in FIG. 1, 4-hydroxy-2,2,6,6-tetramethyl-piperidine-1-oxyl (TEMPOL) has the ability to inhibit the manifestation of clinical signs of EAE in rats. The dosage of TEMPOL used here was 2-6 times higher than the dosages of compounds 6 and 7 below. The experiments were terminated arbitrarily at day 18 post-immunization. The course of EAE in different groups of animals varies. Consequently, recovery time is variable, typically by two or three days.

Compounds 1 and 5 were given orally, daily, to mice induced with EAE in different doses and the results are summarized in Table 3b.

Compounds 6 and 7 were given orally to mice challenged with PLP, at a dose of 50 mg/kg/day, p.o. The results are presented in Table 3a and in FIGS. 2 and 3. The effect of Compound 6 on all parameters of the disease was more pronounced as compared to the control group. The mean maximal score (3.73 vs 5.2), the mean disease duration (2.68 vs 7.9), the mean day of onset (15.2 vs 11.05) and the group mean score (1.27 vs 3.28) were significantly different. The activity of Compound 7 was also pronounced and all parameters of the disease were significantly different as compared to the control group.

The results indicate that both Compounds 6 and 7 may be promising drugs for the inhibition of autoimmune or neurologic disorders in the brain. They both showed a significant beneficial effect on EAE. The treatment with these drugs caused a long delay in the onset of clinical signs which may indicate that the compounds may have an effect on cell activation or proliferation.

TABLE 3a
EAE (single dose daily, 50 mg/kg/day po, PLP challenge, mice)

| | Incidence | Mean | Mean | Mean day | Mean |
|---|---|---|---|---|---|
| group | (# dead) | maximal | disease | of onset | score |
| control | 14/14 | 5.2 ± 0.08 | 7.9 ± 0.4 | 11.05 ± 0.2 | 3.28 ± 0.25 |
| | (4) | | | | |
| Compound 6 | 10/14 | 3.73 ± 0.88* | 2.68 ± 0.73* | 15.2 ± 0.98* | 1.27 ± 0.26* |
| | (4) | | | | |
| Compound 7 | 11/14 | 4.3 ± 0.9** | 4.1 ± 0.84* | 14.8 ± 0.94* | 1.6 ± 0.27* |
| | (4) | | | | |

Results are given as mean ± SEM. Rank sum test, Kruskal-Wallis one way analysis of variance on ranks.
*significantly different as compared to the control group (T test, Mann-Whitney).
**not significant as compared to the control group.

TABLE 3b
EAE (single dose daily, po, mice)

| | | Mean maximal | Mean disease | Mean day of | |
|---|---|---|---|---|---|
| Group | Incidence | score^{a} | duration^{c} | onset^{d} | Mean score^{b} |
| Control | 17/20 | 5 | 5.82 ± 2.3 | 11.1 ± 0.9 | 3.6 ± 0.4 |
| Vehicle | | | | | |
| Compound 1 | 6/8 | 4.2 | 5.57 ± 1.72 | 12.5 ± 0.7 | 3.4 ± 0.2 |
| (50 mg/kg) | | | | | |
| Compound 5 | 18/18 | 4.5 | 6.06 ± 1.7 | 12 ± 0.68 | 3.8 ± 0.35 |
| (12.5 mg/kg) | | | | | |
| Compound 5 | 14/16 | 5 | 5.64 ± 1.55 | 12.9 ± 1.35 | 3.48 ± 0.3 |
| (50 mg/kg) | | | | | |
| Compound 5 | 5/5 | 5 | 4.4 ± 1.7 | 11.8 ± 1.1 | 3.2 ± 0.25 |
| (100 mg/kg) | | | | | |

^{a}Mean maximal score: Maximal score of each mouse in the group/number of mice
^{b}Mean score: all scores exhibited by all mice within a group/number of days
^{c}Mean disease duration: the death score (6) is carried up to the end of observation.
^{d}Mean disease onset (calculated only for mice that develop disease)

2.2 EAE in CSJL Mice—Acute Model
Species, Strain and Supplier

Healthy, nulliparous, non-pregnant female mice of the CSJL/FI strain were obtained from Harlan Animal Breeding Center, Jerusalem, Israel.

The animals weighed about 17-20 g on arrival, and were approximately 7 weeks of age.

The body weights of the animals were recorded on the day of delivery.

Overtly healthy animals were assigned to study groups arbitrarily before treatment commenced.

The mice were individually identified by markings on the body. A color-coded card on each cage gave information including cage number, group number and identification.

TEST Procedures

a. EAE Induction

EAE was induced by injecting the encephalitogenic mixture (emulsion) consisting of MSCH and commercial CFA containing 1 mg/mL Mycobacterium tuberculosis to the foot-pads of the animals. Pertussis toxin was injected intravenously on the day of induction and 48 hours later.

b. Group Assignment

The mice were allocated to the following treatment groups (10 mice/group):

TABLE 4

| | | | | Duration |
|---|---|---|---|---|
| | Treatment | | Administration | of |
| Group | groups | dose/day (*) | Route | treatment |
| 1 | Control | 10 mL/kg × 2* | oral | 30 days |
| 2 | Compound 6 | 50.0 mg/kg | oral | 30 days |
| 3 | Compound 6 | 25.0 mg/kg × 2* | oral | 30 days |
| 4 | Compound 7 | 50.0 mg/kg | oral | 30 days |
| 5 | Compound 7 | 25.0 mg/kg × 2* | oral | 30 days |

*= On weekends, only a single dose of the test articles was given

C. Preparation of Test Solutions of Compound 6 and Compound 7

A 5.0 mg/mL stock solution of the test articles was prepared daily in purified water for dose level of 50.0 mg/kg. The sample was crushed, weighed and dissolved in purified water. The 5.0 mg/mL solution was vortexed and 2.5 mg/mL solution of the test articles was prepared in purified water for dose level of 25.0 mg/kg×2.

d. Test Article Administration

The mice were administered with the respective dose levels of Compound 6 and Compound 7 at volume dose level of 200 μL/mouse. The test formulations were vortexed before dispension in syringe.

These test articles were administered to the respective groups by oral gavage daily for 30 consecutive days, starting from the day of induction until the termination of the study.

The test formulations of 25.0 mg/kg Compound 6 and 25 mg/kg Compound 7 were administered to the treatment groups twice a day except on weekends. On weekends (Fridays and Saturdays) the same concentrations of the test articles were administered once a day.

The vehicle was administered to Group # 1 in a similar manner.

The test formulations of 50.0 mg/kg Compound 6 and 50.0 mg/kg Compound 7 were administered to the treatment groups once a day.

Experimental Observations

a. Morbidity and Mortality

All animals were examined once daily to detect if any is dead or moribund.

b. Clinical Signs

Scoring of EAE clinical signs was initiated from Day 10 post-EAE induction and was continued daily for 20 days. The clinical signs were recorded on observation cards according to a grading system described in the table below.

TABLE 5
Evaluation of the EAE clinical signs.

| Score | Signs | Description |
|---|---|---|
| 0 | Normal behavior | No neurologic signs. |
| 1 | Tail weakness | The mouse tail is limp and |
| | | droops. |
| 2 | Hind legs weakness | Limb paresis, wobbly walk - when |
| | | the mouse walks the hind legs are |
| | | unsteady. |
| 3 | Hind legs paralysis | The mouse can't move it's hind |
| | | legs and it drags them when he |
| | | walks. |
| 4 | Full paralysis | The mouse can't move it's legs at |
| | | all, it looks thinner and |
| | | emaciated. |
| 5 | Death | |

All mice having scores of 1 and above were considered sick.

All animals having a score of 4 were sacrificed on humane grounds.

For calculation purposes, the score of animals that were sacrificed (4) or died (5) was carried forward.

c. Interpretation of Results

Calculation of the Incidence of Disease (Disease Ratio)

The number of sick animals in each group was summed.
The incidence of disease was calculated as
   Calculation of Mean Delay in Onset of Disease

The mean delay in onset of disease expressed in days was calculated by subtracting the mean onset of disease in the control group from the test group.

For calculation purposes, the onset period for a mouse that did not develop EAE during the observation period was considered as 31 days.

Calculation of the Mean Maximal Score and Percent Inhibition

The mean maximal score (MMS) of each group was calculated as
Σ maximal score of each mouse/number of mice in the group.
The percent inhibition was calculated as

$$Percent inhibition = 1 - \frac{( MMS of treated group )}{MMS of control group} \times 100$$

Calculation of the Mean Group Score and Percent Inhibition

The daily scores of each mouse in the test group was summed and the individual mean daily score (IMS) was calculated as
Σ daily score of mouse/observation period (days).

The mean group score (GMS) was calculated as
Σ IMS of each mouse/number of mice in the group.

The percent inhibition (% I) was calculated as

$$( \% I ) = 1 - \frac{GMS of treated grp}{GMS of control grp} \times 100$$

Results

The individual and mean scores and the onset and duration of EAE for each mouse and a figure of the EAE clinical profile for each group are presented below.

A summary table of the mortality rate, % incidence, mean maximal score, group mean score and mean duration of disease is shown below.

TABLE 6
Summary table

| | | | | | MMS | | GMS | | Mean |
|---|---|---|---|---|---|---|---|---|---|
| Group | | | | % | | % | | % | Duration |
| No. | Treatment | Mortality | incidence | inhibition 1 | value | inhibition 2 | value | inhibition 3 | (days) |
| 1 | Control | 6/10 | 10/10 | — | 4.1 ± 1.2 | — | 3.2 ± 1.1 | — | 17.6 ± 1.8 |
| 2 | Compound 6 | 5/10 | 7/10 | 30.0% | 3.0 ± 2.3 | 26.8% | 2.2 ± 1.8 | 31.3% | 11.3 ± 8.5 |
| | (50.0 mg/kg) | | | | | | | | |
| 3 | Compound 6 | 4/10 | 7/10 | 30.0% | 2.7 ± 2.2 | 34.1% | 1.9 ± 1.9 | 40.6% | 11.1 ± 8.6 |
| | (25.0 mg/kg × 2) | | | | | | | | |
| 4 | Compound 7 | 7/10 | 7/10 | 30.0% | 3.5 ± 2.4 | 14.6% | 2.7 ± 1.9 | 15.6% | 12.7 ± 8.8 |
| | (50.0 mg/kg) | | | | | | | | |
| 5 | Compound 7 | 8/10 | 8/10 | 20.0% | 4.0 ± 2.1 | 2.4% | 2.9 ± 1.6 | 9.4% | 13.9 ± 7.5 |
| | (25.0 mg/kg × 2) | | | | | | | | |

Discussion

EAE (Experimental Allergic Encephalomyelitis) is an accepted animal model of autoimmune disorders such as multiple sclerosis (see Tisch, R. and McDevitt, H. O. Proc. Natl. Acad. Sci. USA (1994) 91: 437-438 and references cited therein). The acute model for EAE (Section 4 above) can be utilized to measure the effects of the compounds of the invention on inflammation (E. Betteli and J. B. Nicholson, Arch. Immun. Ther. Exp. (Warsz.), 48(5), 389-398 (2000)). As such, the results presented above suggest that the compounds of the present invention would be effective drugs for treating inflammation and autoimmune disorders such as MS in humans.

In addition, oxidative stress has been implicated in a variety of neurologic or autoimmune disorders, as discussed in the background of the invention (See, for example, M. P. Mattson et al., J. Neurosci. Res. (1997) 49: 681). As illustrated by the examples above, the compounds of the present invention are effective antioxidants and free radical scavengers. This data, evaluated in light of the EAE experimental data presented above, suggests that the compounds of the present invention would be effective treatments for a variety of neurologic or autoimmune disorders which involve oxidative stress.

1. A compound having the structure: 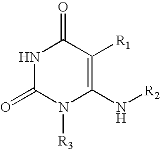
wherein,
R₁ is H, NH₂, NH—(C₁-C₄)alkyl, or N—[(C₁-C₄)alkyl]₂;
R₂ and R₃ are each independently H, (C₁-C₄)alkyl, or 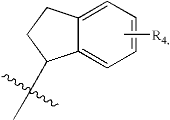
wherein R₄ is H, (C₁-C₄)alkyl, halogen, hydroxy, (C₁-C₁₀)alkoxy, cyano, nitro, —NR₅R₆, or —OCONR₇R₈;
wherein R₅ and R₆ are each independently H, or a substituted or unsubstituted (C₁-C₄)alkyl; and
wherein R₇ and R₈ are each independently H, or substituted or unsubstituted (C₁-C₄)alkyl, or (C₁-C₁₀)aryl; and
wherein only one of R₂ and R₃ is H, or an enantiomer, or a tautomer, or a pharmaceutically acceptable salt thereof.

2. The compound of claim 1, wherein
R₁ is H or NH₂;
R₂ and R₃ are each independently H, or 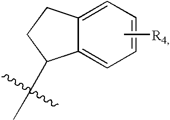
wherein R₄ is H, (C₁-C₄)alkyl, halogen, hydroxy, (C₁-C₁₀)alkoxy, cyano, nitro, —NR₅R₆, or —OCONR₇R₈;
wherein R₅ and R₆ are each independently H, or a substituted or unsubstituted (C₁-C₄) alkyl; and
wherein R₇ and R₈ are each independently H, or substituted or unsubstituted (C₁-C₄)alkyl, or (C₁-C₁₀)aryl; and
wherein only one of R₂ and R₃ is H.

3. The compound of claim 2, having the structure: 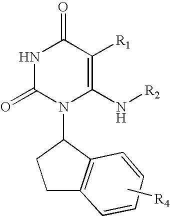
wherein
R₁ is H or NH₂;
R₂ is H; and
R₄ is H, (C₁-C₄) alkyl, halogen, hydroxy, (C₁-C₁₀)alkoxy, cyano, nitro, —NR₅R₆, or —OCONR₇R₈;
wherein R₅ and R₆ are each independently H, or a substituted or unsubstituted (C₁-C4)alkyl; and
wherein R₇ and R₈ are each independently H, or substituted or unsubstituted (C₁-C₄)alkyl, or (C₁-C₁₀)aryl.

4. The compound of claim 3, having the structure:

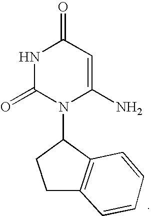

5. The compound of claim 3, having the structure:

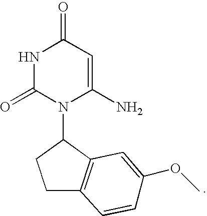

6. The compound of claim 3, having the structure:

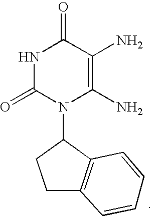

7. The compound of claim 2, having the structure 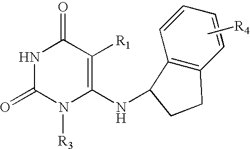
wherein
R₁ is H or NH₂;
R₃ is H; and
R₄ is H, (C₁-C₄)alkyl, halogen, hydroxy, (C₁-C₁₀)alkoxy, cyano, nitro, —NR₅R₆, or —OCONR₇R₈;
wherein R₅ and R₆ are each independently H, or a substituted or unsubstituted (C₁-C₄)alkyl; and
wherein R₇ and R₈ are each independently H, or substituted or unsubstituted (C₁-C₄)alkyl, or (C₁-C₁₀)aryl.

8. The compound of claim 7, having the structure:

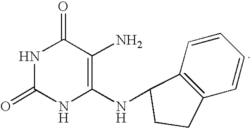

9. The compound of claim 7, having the structure:

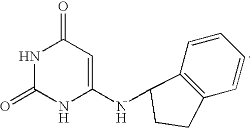

10. The compound of claim 7, having the structure:

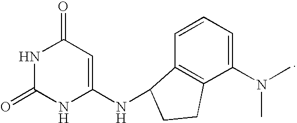

11. The hydrochloride salt of the compound of claim 10.

12. The compound of claim 7, having the structure:

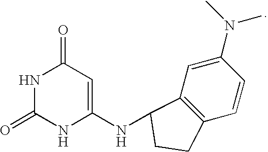

13. The hydrochloride salt of the compound of claim 12.

14. The compound of claim 7, having the structure:

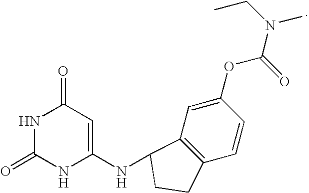

15. The compound of claim 7, having the structure:

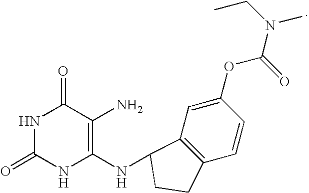

16. A compound having the structure: 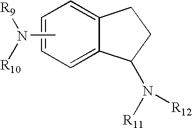
wherein,
R₉ and R₁₀ are each independently a substituted or unsubstituted (C₁-C₄)alkyl; and
R₁₁ and R₁₂ are each independently H or a substituted or unsubstituted (C₁-C₄)alkyl, or an enantiomer, or a tautomer, or a pharmaceutically acceptable salt thereof.

17. The compound of claim 16, wherein R₉ and R₁₀ are both methyl, and R₁₁ and R₁₂ are both H.

18. A method of treating a subject suffering from a neurologic disorder or an autoimmune disorder, comprising administering to the subject a therapeutically effective amount of the compound of claim 1 so as to thereby treat the subject.

19-25. (canceled)

26. A method of treating a subject afflicted with an inflammatory disorder caused by the presence of reactive oxidative species, comprising administering to the subject a therapeutically effective amount of the compound of claim 1 so as to thereby treat the subject.

27-29. (canceled)

30. A method of preventing the oxidation of lipids, proteins or deoxyribonucleic acid in a cell, comprising contacting the cell with the compound of claim 1.

31. A method of preventing lysis of human red blood cells by O₂ radicals, comprising contacting the cells with the compound of claim 1.

32. A pharmaceutical composition comprising the compound of claim 1 and a pharmaceutically acceptable carrier.

33. A process for the manufacture of a pharmaceutical composition comprising admixing the compound of claim 1 with a pharmaceutically acceptable carrier.

34. (canceled)

35. A process for manufacturing a compound having the structure: 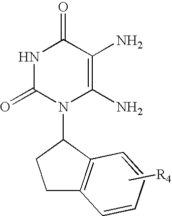
wherein R₄ is H, (C₁-C₄)alkyl, halogen, hydroxy, (C₁-C₁₀)alkoxy, cyano, nitro, —NR₅R₆, or —OCONR₇R₈;
wherein R₅ and R₆ are each independently H, or a substituted or unsubstituted (C₁-C₄)alkyl; and
wherein R₇ and R₈ are each independently H, or substituted or unsubstituted (C₁-C₄)alkyl, or (C₁-C₁₀)aryl,
comprising reacting 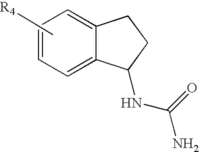
with a cyclization agent, so as to produce the compound.

36-38. (canceled)

39. A process of manufacturing a compound having the structure: 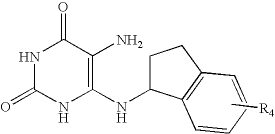
wherein R₄ is H, (C₁-C₄)alkyl, halogen, hydroxy, (C₁-C₁₀)alkoxy, cyano, nitro, —NR₅R₆, or —OCONR₇R₈;
wherein R₅ and R₆ are each independently H, or a substituted or unsubstituted (C₁-C₄)alkyl; and
wherein R₇ and R₈ are each independently H, or substituted or unsubstituted (C₁-C₄)alkyl, or (C₁-C₁₀)aryl,
comprising reacting 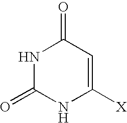
wherein X is Cl or NH₂, with 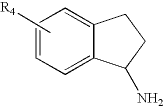
in a high boiling solvent to produce the compound.

40-42. (canceled)

43. A process of manufacturing the compound of claim 17, comprising the steps of:
(a) reacting 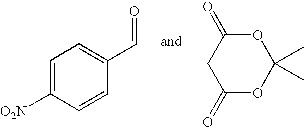
in the presence of triethylammonium formate reagent to produce 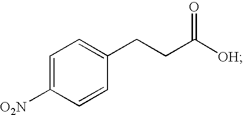
(b) reducing the product of step (a) with Pd/C in the presence of triethyl amine and formic acid to produce 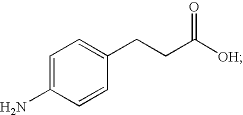
(c) reacting the product of step (b) with acetic anhydride to produce 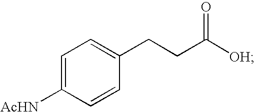
(d) reacting the product of step (c) with a cyclization agent to produce 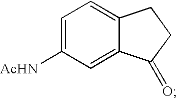
(e) removing the acetyl group of the product of step (d) by reacting it with an acid to produce 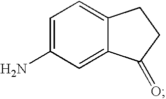
(f) reacting the product of step (e) with paraformaldehyde and hydrogen over a palladium catalyst to produce

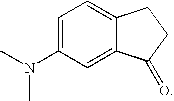

44-48. (canceled)

49. A process for manufacturing a compound having the structure 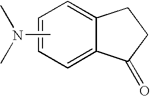
comprising reacting a compound having the structure 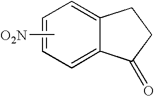
with paraformaldehyde and hydrogen over a palladium catalyst to produce the compound.

50-51. (canceled)

52. The compound of claim 16 or 17 having the structure

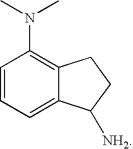

53. A process for manufacturing the compound of claim 52 comprising reacting a compound having the structure 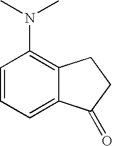
with sodium cyanoborohydride and ammonium acetate in the presence of solvent to produce

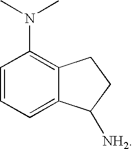

54. A process for manufacturing the compound of claim 52 comprising:
(1) reacting a compound having the structure 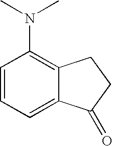
with hydroxylamine to produce 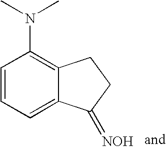
(2) reducing the product of step (1) with a reducing agent to produce

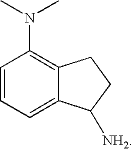

55-69. (canceled)
